(19) United States
(12) Patent Application Publication
(10) Pub. No.: US 2014/0005483 A1
(43) Pub. Date: Jan. 2, 2014

(54) LIGHT SOURCE APPARATUS AND ENDOSCOPE SYSTEM

(71) Applicant: FUJIFILM Corporation, Tokyo (JP)

(72) Inventors: Eiji OHASHI, Ashigarakami-gun (JP); Yoshinori MORIMOTO, Ashigarakami-gun (JP); Toshiyuki INOUE, Ashigarakami-gun (JP)

(21) Appl. No.: 13/932,291

(22) Filed: Jul. 1, 2013

(30) Foreign Application Priority Data
Jul. 2, 2012 (JP) 2012-148286

(51) Int. Cl. A61B 1/00; F21V 33/00

(52) U.S. Cl. CPC A61B 1/00195; F21V 33/0068
USPC 600/162; 362/574

(57) ABSTRACT

A light source apparatus for supplying a light guide device incorporated in an endoscope with a light beam is provided. A light emitting device of semiconductor, for example, laser diode generates the light beam. A light homogenizer homogenizes irradiance distribution of the light beam in a radial direction. A short focus lens is disposed between the light homogenizer and the light guide device, for enlarging a divergence angle of the light beam. Furthermore, the light homogenizer is a transparent light guide rod disposed to extend in an optical axis direction of the light beam. A diameter of the light homogenizer is constant in an optical axis direction thereof. The diameter of the light homogenizer is equal to or less than a diameter of the short focus lens.

### BACKGROUND OF THE INVENTION

1. Field of the Invention

The present invention relates to a light source apparatus and an endoscope system. More particularly, the present invention relates to a light source apparatus and an endoscope system, in which an area of lighting with a light beam from light source units can be enlarged, and a difference in a light amount between the center and periphery can be reduced.

2. Description Related to the Prior Art

An endoscope system for diagnosis of a body cavity is widely used in a medical field. The endoscope system includes an endoscope, a light source apparatus, and a processing apparatus. The endoscope includes an elongated tube and a tip device. The elongated tube is entered in the body cavity. The tip device is disposed at a distal end of the elongated tube, images an object in the body cavity, and outputs an image signal. The light source apparatus supplies the endoscope with light for illumination. The processing apparatus processes the image signal output by the endoscope. There are lighting windows and an imaging window formed in the tip device. The lighting windows emit light to the object. The imaging window receives object light from the object for imaging. A light guide device is incorporated in the elongated tube, and has a fiber bundle in which plural optical fibers are bundled. The light guide device guides the light generated by the light source apparatus in the distal direction to the lighting windows in the tip device.

Also, JP-A 2011-041758 discloses a type of the light source apparatus in which a light emitting device of semiconductor is used as the light source in place of a xenon lamp or halogen lamp, for example, a laser diode (LD) and light-emitting diode (LED).

The light emitting device of semiconductor emits a light beam which travels characteristically to spread in a conical form from an emission point. The light beam of the light emitting device travels with a smaller divergence angle than that of a xenon lamp or halogen lamp, because directionality of the light beam is higher.

In FIG. 46, intensity distribution of a light beam from a laser diode as a light source is illustrated. In a graph of the intensity distribution, the horizontal axis represents an output ray angle θi. The vertical axis represents intensity I. The intensity I is a radiation flux (in lumen) per unit solid angle (in sr or steradian). A unit of the intensity I is lumen/sr. A divergence angle θ of the laser diode is expressed as a half width at a half maximum (HWHM) which is a half of a full width on the condition of indicating a half relative to the maximum of the intensity I. A specific value of the divergence angle θ of the laser diode is approximately 10 degrees as a half width at half maximum, namely approximately 20 degrees as a full width at half maximum.

The intensity distribution of the laser diode is a Gaussian distribution in which the intensity I abruptly drops from a peak of a curve with a steep inclination. In contrast, the intensity distribution of the xenon lamp or halogen lamp is a top-hat distribution in which a peak of a curve is relatively flat as an original position (output ray angle θi=0), and a high angle component with a large value of the output ray angle θi drops slowly. In the Gaussian distribution, the divergence angle θ is smaller than the top-hat distribution, because of a more abrupt drop in the high angle component. The divergence angle θ of a LED and laser diode is smaller than that of the xenon lamp and halogen lamp. Among those, the divergence angle θ of the laser diode is still smaller than that of the LED.

The divergence angle of a light beam is maintained during passage in the light guide device in the endoscope. If the divergence angle incident upon the light guide device is small, the divergence angle of an exiting light beam from the light guide device is also small. An object of interest is illuminated by the light beam with the small the divergence angle. There occurs a problem in that an area of lighting with the light beam is small on the object of interest. The area of lighting is preferably large for the purpose of maintaining a large area of view for endoscopic imaging. Furthermore, a problem of a considerably large difference in the light amount between the center and periphery is serious in irradiance distribution in a region of the object of interest, because the peripheral light amount is extremely smaller than a center light amount. Visual recognition of the object of interest will be difficult on the condition of this large difference in the light amount.

JP-A 2011-041758 and other technical documents do not disclose a technique for solving the problem described above.

### SUMMARY OF THE INVENTION

In view of the foregoing problems, an object of the present invention is to provide a light source apparatus and an endoscope system, in which an area of lighting with a light beam from light source units can be enlarged, and a difference in a light amount between the center and periphery can be reduced.

In order to achieve the above and other objects and advantages of this invention, a light source apparatus for supplying a light guide device incorporated in an endoscope with a light beam is provided. A light emitting device of semiconductor generates the light beam. A light homogenizer homogenizes irradiance distribution of the light beam in a radial direction. A lens is disposed between the light homogenizer and the light guide device, for enlarging a divergence angle of the light beam.

The light homogenizer is a transparent light guide rod disposed to extend in an optical axis direction of the light beam.

A diameter of the light homogenizer is constant in an optical axis direction thereof.

The diameter of the light homogenizer is equal to or less than a diameter of the lens.

The diameter of the light homogenizer is equal to a diameter of the lens.

The lens is a short focus lens.

The light emitting device is a laser diode.

The light beam is narrow band light of a wavelength range of blue.

A beam profile of the light beam is elliptical. The light homogenizer includes a beam shaping device for beam shaping of the light beam of the elliptical beam profile into a circular beam profile.

The light homogenizer includes an entrance end face for receiving incidence of the light beam from the light emitting device. An exit end face emits the light beam toward the lens. A reflective interface is disposed to extend from the entrance end face to the exit end face, for internally reflecting the light beam and constituting the beam shaping device.

The light beam includes first and second components located in respectively radial directions along major and minor axes of the elliptical form. The reflective interface twists at least one of the first and second components about the optical axis direction by reflection.

In another preferred embodiment, the light beam includes first and second components located in respectively radial directions along major and minor axes of the elliptical form. The reflective interface includes a first portion for receiving incidence of at least one of the first and second components in a non-normal direction.

The light homogenizer includes an entrance end face for receiving incidence of the light beam from the light emitting device. An exit end face emits the light beam toward the lens. A reflective interface is disposed to extend from the entrance end face to the exit end face, for internally reflecting the light beam.

The light homogenizer is cylindrical and extends in the optical axis direction.

In still another preferred embodiment, the light homogenizer is in a form of a polygonal prism extending in the optical axis direction, and the reflective interface includes a plane.

The reflective interface has a curved surface at least partially.

The light beam includes first and second components located in respectively radial directions along major and minor axes of the elliptical form. At least one of the first and second components is reflected by the curved surface at a reflection point, and travels in a direction non-normal to a tangential line of the curved surface at the reflection point.

A form of the light homogenizer in a cross section transverse to the optical axis direction is circular, eccentrically looped or elliptical, and a center of the form in the cross section is offset from an emission center of the light emitting device.

Also, an endoscope system including an endoscope having a light guide device inside, and a light source apparatus for supplying the light guide device with a light beam is provided. The light source apparatus includes a light emitting device of semiconductor for generating the light beam. A light homogenizer homogenizes irradiance distribution of the light beam in a radial direction. A lens is disposed between the light homogenizer and the light guide device, for enlarging a divergence angle of the light beam.

Consequently, an area of lighting with a light beam from light source units can be enlarged, and a difference in a light amount between the center and periphery can be reduced, because of effect of the lens in combination with the light homogenizer for enlarging the divergence angle of the light beam.

### BRIEF DESCRIPTION OF THE DRAWINGS

The above objects and advantages of the present invention will become more apparent from the following detailed description when read in connection with the accompanying drawings, in which:

FIG. 1 is a perspective illustrating an endoscope system;

FIG. 2 is a front elevation illustrating a tip device of an endoscope;

FIG. 3 is a block diagram schematically illustrating the endoscope system;

FIG. 4 is a graph illustrating spectral distribution of light;

FIG. 5 is a graph illustrating an absorption spectrum of hemoglobin;

FIG. 6 is a graph illustrating a scattering coefficient of tissue;

FIG. 7 is a graph illustrating spectral distribution of a color micro filter;

FIG. 8A is a timing chart illustrating lighting and imaging with the light;

FIG. 8B is a timing chart illustrating lighting and imaging in a vessel enhancement mode;

FIG. 8C is a timing chart illustrating lighting and imaging in an oxygen saturation monitoring mode;

FIG. 9A is a flow chart illustrating image processing in a normal imaging mode;

FIG. 9B is a flow chart illustrating image processing in the vessel enhancement mode;

FIG. 9C is a flow chart illustrating image processing in the oxygen saturation monitoring mode;

FIG. 10 is a perspective illustrating an optical routing device and a light source unit;

FIG. 11 is an explanatory view in a section illustrating arrangement of optical fibers;

FIG. 12 is an explanatory view in a section illustrating a photo sensor for light measurement and an output adjusting device;

FIG. 13 is a perspective illustrating a first light source unit;

FIG. 14 is an explanatory view in a section illustrating a divergence angle corrector of the first light source unit;

FIG. 15 is a perspective illustrating a second light source unit;

FIG. 16 is an explanatory view in a section illustrating a divergence angle corrector of the second light source unit;

FIGS. 17A-17E are graphs illustrating intensity distribution and irradiance distribution at points in the structure of FIG. 22;

FIG. 18 is an explanatory view in a side elevation illustrating spot diameters of the first and second light source units;

FIG. 19 is an explanatory view in a section illustrating Comparison 1;

FIGS. 20A-20E are graphs illustrating intensity distribution and irradiance distribution according to Comparison 1;

FIG. 21 is an explanatory view in a section illustrating Comparison 2;

FIGS. 22A-22E are graphs illustrating intensity distribution and irradiance distribution according to Comparison 2;

FIG. 23 is an explanatory view in a section illustrating diameters of a hemispherical lens and the light homogenizer;

FIG. 24 is a perspective illustrating another preferred light homogenizer of a form of a hexagonal prism;

FIG. 25 is a perspective illustrating a shape of a light beam;

FIG. 26 is a graph illustrating intensity distribution of the light beam;

FIG. 27 is an explanatory view in a cross section illustrating a relationship between the light homogenizer and the light beam;

FIG. 28 is a perspective illustrating a locus of a component of a minor axis in the light beam;

FIG. 29 is an explanatory view in a cross section illustrating reflection and twist of the component of the minor axis;

FIG. 30 is a perspective illustrating a locus of a component of a major axis in the light beam;

FIG. 31 is an explanatory view in a cross section illustrating reflection and twist of the component of the major axis;

FIG. 32 is an explanatory view in a cross section illustrating reflection and twist of an intermediate component in the light beam;

FIG. 33 is an explanatory view in a cross section illustrating light components without twist about an optical axis;

FIG. 34 is an explanatory view in a cross section illustrating shapes of an incident light beam and an exiting light beam;

FIG. 35 is a graph illustrating intensity distribution of the exiting light beam;

FIG. 36 is an explanatory view in a cross section illustrating still another preferred light homogenizer in which the structure of FIG. 27 is oriented differently;

FIG. 37 is an explanatory view in a cross section illustrating another preferred light homogenizer in which the structure of FIG. 30 is oriented differently;

FIG. 38 is an explanatory view in a cross section illustrating still another preferred light homogenizer oriented in a non-concentric manner;

FIG. 39 is an explanatory view in a cross section illustrating another preferred light homogenizer of a form of a quadrilateral prism;

FIG. 40 is an explanatory view in a cross section illustrating still another preferred light homogenizer in which the structure of FIG. 39 is oriented differently;

FIG. 41 is an explanatory view in a cross section illustrating another preferred light homogenizer in which the structure of FIG. 39 is oriented horizontally (normally);

FIG. 42 is an explanatory view in a cross section illustrating still another preferred light homogenizer of a form of a triangular prism;

FIG. 43 is a perspective illustrating another preferred light homogenizer of a cylindrical form;

FIG. 44 is an explanatory view in a cross section illustrating an entrance end face of the light homogenizer;

FIG. 45 is an explanatory view in a cross section illustrating still another preferred light homogenizer in which the structure of FIG. 44 is oriented without offsetting;

FIG. 46 is a graph illustrating intensity distribution of the laser diode.

### DETAILED DESCRIPTION OF THE PREFERRED EMBODIMENT(S) OF THE PRESENT INVENTION

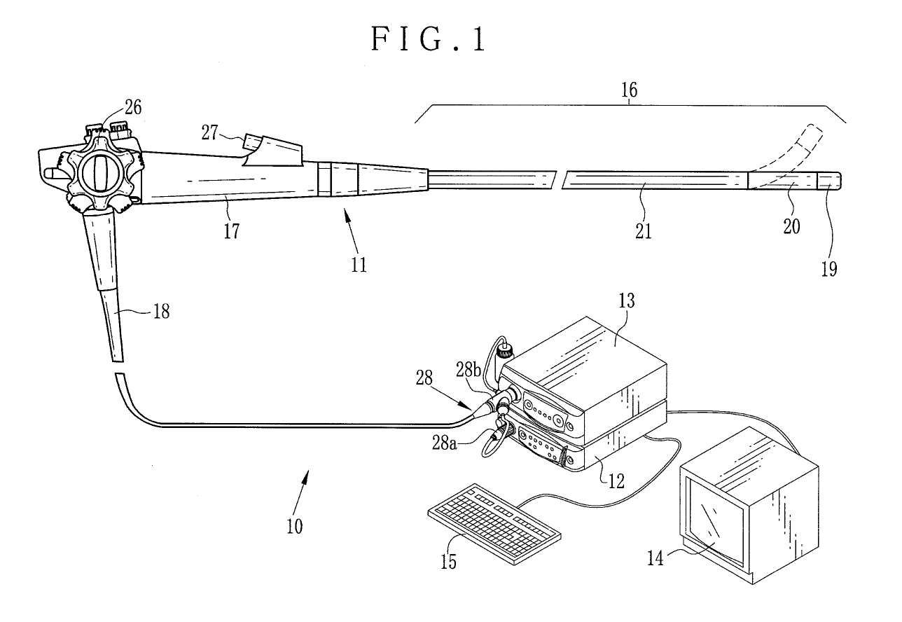

In FIG. 1, an endoscope system 10 includes an endoscope 11, a processing apparatus 12, a light source apparatus 13 and a monitor display panel 14. The endoscope 11 images an object in a body cavity, and generates an image signal. The processing apparatus 12 creates an object image according to the image signal. The light source apparatus 13 supplies the endoscope 11 with light for illuminating the object. The display panel 14 displays the image. An input interface 15 (console unit) for the processing apparatus 12 includes various elements such as a keyboard, mouse and the like.

The endoscope system 10 is operable in a normal imaging mode (color imaging mode) and a special imaging mode. In the normal imaging mode, an object of interest is imaged with white light. In the special imaging mode, special light is used to image blood vessels in a region of interest. In the special imaging mode, a pattern of the blood vessels or an oxygen saturation level is recognized to diagnose a tumor between benign and malignant conditions. An example of the special light is narrowband light with a high wavelength band because of high absorbance in blood hemoglobin. Examples of the special imaging mode include a vessel enhancement mode and an oxygen saturation monitoring mode. In the vessel enhancement mode, a vessel-enhanced image in which the vessels are enhanced is output and displayed. In the oxygen saturation monitoring mode, a special image in which the oxygen saturation level (SO2 level) of the blood hemoglobin is output and displayed.

The endoscope 11 includes an elongated tube 16, a handle 17 and a universal cable 18. The elongated tube 16 is entered in a gastrointestinal tract of a body. The handle 17 is disposed on a proximal end of the elongated tube 16. The universal cable 18 extends between the handle 17 and the processing apparatus 12 and the light source apparatus 13 for connection.

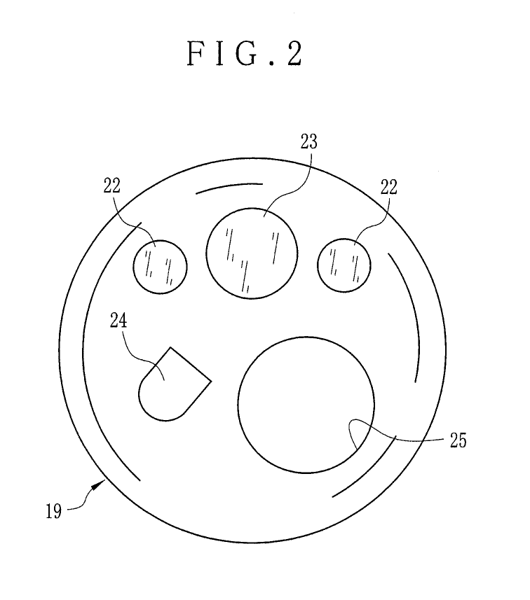

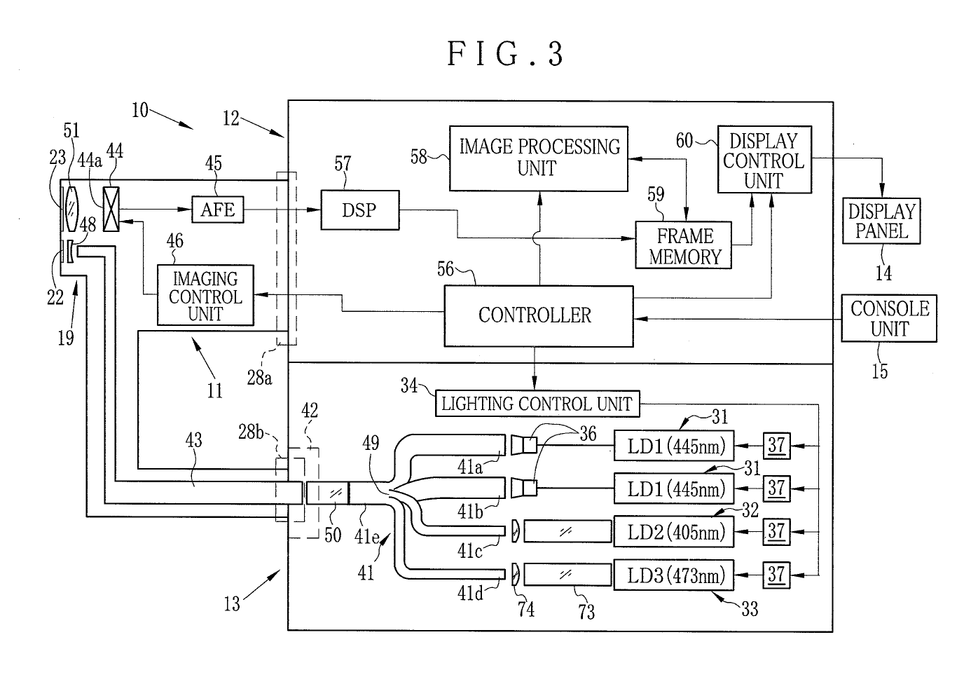

The elongated tube 16 includes a tip device 19, a steering device 20 and a flexible tube device 21 arranged in a proximal direction. In FIG. 2, a distal surface of the tip device 19 has lighting windows 22, an imaging window 23, a nozzle spout of a fluid supply nozzle 24 for air and water, and a distal instrument opening 25. The lighting windows 22 apply light to an object in a body cavity. The imaging window 23 receives object light reflected by the object. The fluid supply nozzle 24 ejects air and/or water for cleaning the imaging window 23. The distal instrument opening 25 causes a medical instrument to protrude, such as a forceps, electrocautery device and the like. An imaging unit 44 is disposed behind the imaging window 23 together with a lens system for image forming. See FIG. 3.

The steering device 20 includes a plurality of link elements connected serially with one another. Steering wheels 26 are mounted on the handle 17, and rotated to steer the steering device 20 up and down and to the right and left. The tip device 19 is directed by bending the steering device 20 as desired by a doctor or operator. The flexible tube device 21 is so flexible as to enter a body cavity in a tortuous form smoothly, such as an esophagus, intestines and the like. The elongated tube 16 contains a communication line and a light guide device 43 of FIG. 3. The communication line transmits a drive signal for driving the imaging unit 44, and an image signal output by the imaging unit 44. The light guide device 43 directs light supplied by the light source apparatus 13 to the lighting windows 22.

A proximal instrument opening 27 is formed in the handle 17 for entry of the medical instrument. Also, the handle 17 has the steering wheels 26, a fluid supply button, a recording button and the like. The fluid supply button is depressible for supply of water and/or air. The recording button is depressible for recording a still image.

The universal cable 18 has the communication line, the light guide device 43 and the like extending from the elongated tube 16. A proximal connector 28 is mounted on a proximal end of the universal cable 18 on the side of the processing apparatus 12 and the light source apparatus 13. The proximal connector 28 is in a composite form and includes a first connection plug 28a for communication and a second connection plug 28b for lighting. An end portion of the communication line is contained in the first connection plug 28a, which is connectively coupled with the processing apparatus 12. An entrance end portion of the light guide device 43 is contained in the second connection plug 28b, which is connectively coupled with the light source apparatus 13.

In FIG. 3, the light source apparatus 13 includes first light source units 31 or modules, a second light source unit 32 or module, a third light source unit 33 or module, and a lighting control unit 34. The light source units 31-33 emit light of wavelengths different between those. The lighting control unit 34 controls the light source units 31-33. The lighting control unit 34 controls a sequence of driving and synchronization of the elements in the light source apparatus 13.

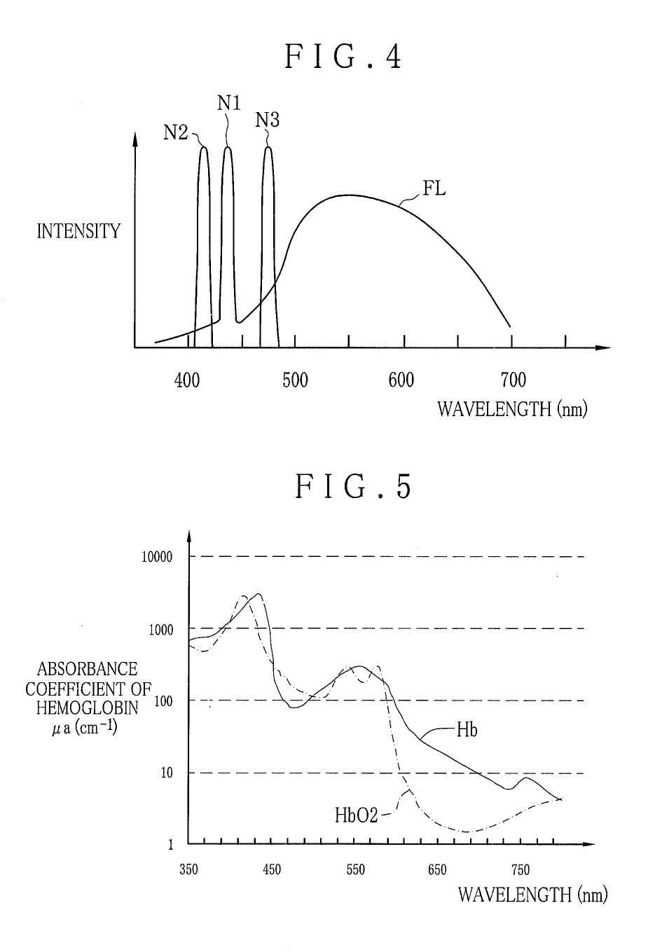

The light source units 31-33 have laser diodes LD1-LD3 for emitting narrow band light of a predetermined wavelength, as light emitting elements of semiconductor. In FIG. 4, the laser diode LD1 emits narrow band light N1 with a center wavelength of 445 nm in a limited band of 440 plus or minus 10 nm in the blue range. The laser diode LD2 emits narrow band light N2 with a center wavelength of 405 nm in a limited band of 410 plus or minus 10 nm in the blue range. The laser diode LD3 emits narrow band light N3 with a center wavelength of 473 nm in a limited band of 470 plus or minus 10 nm in the blue range. Available examples of the laser diodes LD1-LD3 are an InGaN type, InGaNAs type, GaNAs type and the like. A preferable type of the laser diodes LD1-LD3 is a broad area type of which a stripe width (width of waveguide) is large for a structure of high output.

The first light source units 31 emit white light for normal imaging. Phosphor 36 is provided in the first light source units 31 in combination with the laser diode LD1. In FIG. 4, the phosphor 36 is excited by the narrow band light N1 of 445 nm in a blue range emitted by the laser diode LD1, and emits fluorescence FL in a wavelength band from green to red. The phosphor 36 partially absorbs the narrow band light N1 to emit the fluorescence FL, and causes the remainder of the narrow band light N1 to pass. The transmitted component of the narrow band light N1 is diffused by the phosphor 36. The transmitted component is mixed with the fluorescence FL to obtain white light. Examples of the phosphor 36 are a YAG type, BAM type (BgMgAl₁₀O₁₇ type) and the like. The number of the first light source units 31 is two for acquiring a large light amount of white light.

The second light source unit 32 illuminates for the purpose of blood vessel enhancement. In FIG. 5, an absorption spectrum of blood hemoglobin is illustrated. An absorbance coefficient μa in blood has dependency to the wavelength, rises abruptly in a wavelength band under 450 nm, and comes to a first peak at a wavelength near to 405 nm. Also, the absorbance coefficient comes to a second peak at a wavelength of 530-560 nm being lower than the first peak. When light of a wavelength with a high absorbance coefficient μa is applied to an object of interest, an image with clearly high contrast between vessels and other tissue thereabout can be formed because of characteristically high absorption in the vessels.

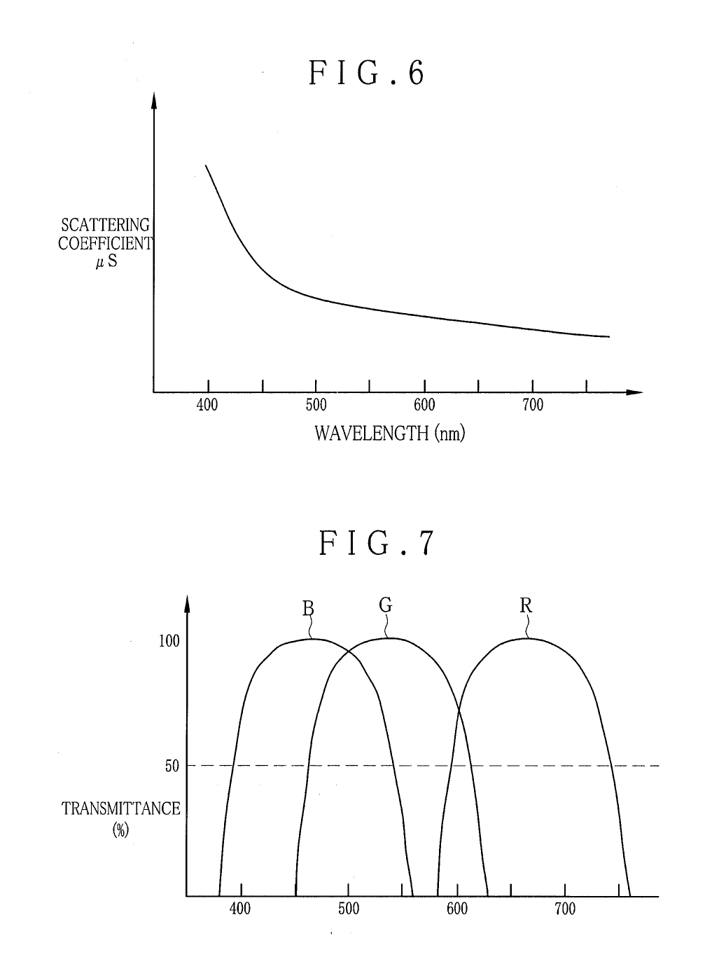

In FIG. 6, a scattering property of tissue for light has dependency to a wavelength. A scattering coefficient μS increases according to shortness of the wavelength. The scattering influences to a penetration depth of light into the tissue of a body. According to highness of the scattering, light reflected near to the surface of the mucosa of the tissue increases, and light reaching the moderately deep or deep layer decreases. Thus, the penetration depth decreases according to the decrease of the wavelength, and increases according to the increase of the wavelength. Wavelengths of light for vessel enhancement are selected in consideration of absorption of hemoglobin, and the scattering property of tissue for light.

The narrow band light N2 of 405 nm emitted by the second light source unit 32 has a small penetration depth, and used for emphasizing surface vessels because of high absorption in the surface vessels. The surface vessels can be imaged with high contrast in a display image by use of the narrow band light N2. Also, a green component in white light emitted by the first light source units 31 is used for emphasizing deep vessels and moderately deep vessels. In the absorption spectrum of FIG. 5, the absorbance coefficient changes more gradually in the green region of 530-560 nm than in the blue region of 450 nm or lower. A band of the light for deep vessels and moderately deep vessels does not need to be so narrow as a band of the blue light. As will be described later, a green component separated from the white color by a micro color filter of green in the imaging unit 44 is used.

The third light source unit 33 is for oxygen saturation monitoring. In FIG. 5, an absorption spectrum Hb is according to the deoxyhemoglobin without bonding to oxygen. An absorption spectrum HbO2 is according to the oxyhemoglobin bonded to oxygen. The deoxyhemoglobin and oxyhemoglobin have absorption properties different from one another. There occurs a difference in the absorbance coefficient μa except for an isosbestic point (intersection point between the spectra Hb and HbO2) of an equal absorbance coefficient μa. Reflectivity is changed upon a change of the oxygen saturation level even in application of light of an equal intensity and equal wavelength, because of the difference in the absorbance coefficient μa. In the oxygen saturation monitoring mode, the narrow band light N3 emitted by the third light source unit 33 of a wavelength 473 nm with a difference in the absorbance coefficient μa is used for measuring the oxygen saturation level.

Drivers 37 are controlled by the lighting control unit 34 to turn on and off the laser diodes LD1-LD3 and control their light amounts. To this end, the lighting control unit 34 generates drive pulses to drive the laser diodes LD1-LD3. A duty factor of the drive pulses is controlled according to the PWM (pulse width modulation) control to change a drive current. Note that the control of the drive current (or power) for the laser diodes LD1-LD3 can be the PAM (pulse amplitude modulation) control to change the amplitude instead of the PWM control.

An optical routing device 41 for guiding light is disposed downstream of the light source units 31-33. The optical routing device 41 aligns optical paths from the light source units 31-33 in one direction. As an entrance end of the light guide device 43 of the endoscope 11 is single, the optical paths from the light source units 31-33 are aligned by the optical routing device 41 for supply of light from those to the endoscope 11. The optical routing device 41 has four input fiber ends 41a, 41b, 41c and 41d (branch waveguides), a routing section 49, and an output fiber end 41e (end waveguide).

The first light source units 31 are opposed to the input fiber ends 41a and 41b of the optical routing device 41. The second and third light source units 32 and 33 are opposed to respectively the input fiber ends 41c and 41d of the optical routing device 41.

A receptacle connector 42 or mating coupler is disposed for connection with the second connection plug 28b of the endoscope 11. The output fiber end 41e of the optical routing device 41 is positioned near to the receptacle connector 42. A light homogenizer 50 is disposed downstream of the output fiber end 41e. Light from the light source units 31-33 upon entry in the optical routing device 41 is passed through the light homogenizer 50 and supplied to the light guide device 43 of the endoscope 11 disposed in the second connection plug 28b.

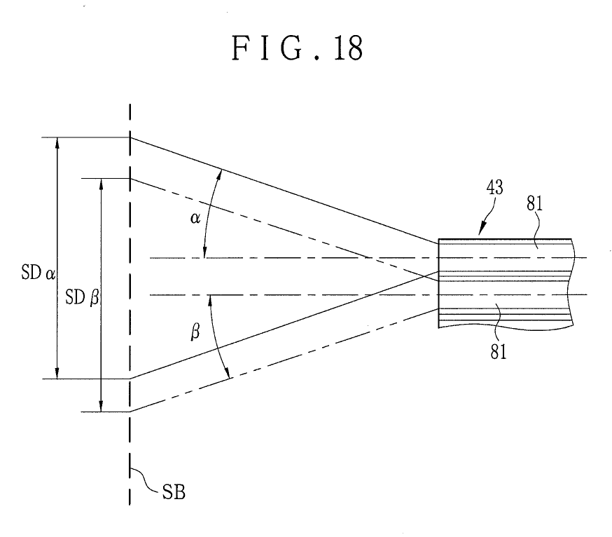

The endoscope 11 includes an analog processing unit 45 (AFE) and an imaging control unit 46 in addition to the light guide device 43 and the imaging unit 44. The light guide device 43 is a fiber bundle including plural optical fibers (referred to by a reference numeral 81 in FIG. 18) bundled together. When the proximal connector 28 is connectively coupled to the light source apparatus 13, an entrance end face of the light guide device 43 is opposed to an exit end face of the light homogenizer 50. The exit end face of the light guide device 43 is branched in two portions upstream of the lighting windows 22, for guiding light to both of the lighting windows 22.

An illumination lens 48 is disposed behind the lighting windows 22. Light generated by the light source apparatus 13 is guided by the light guide device 43 to the illumination lens 48, and emitted by the lighting windows 22 toward a region of interest. An example of the illumination lens 48 is a concave lens which increases a divergence angle of the light output by the light guide device 43. It is possible to apply the light to the region of interest in an enlarged manner.

An objective lens system 51 and the imaging unit 44 are disposed behind the imaging window 23. Object light reflected by the object becomes incident upon the lens system 51 through the imaging window 23. An imaging surface 44a is disposed on the imaging unit 44, where the light is focused by the lens system 51.

The imaging unit 44 is a CCD or CMOS image sensor. The imaging surface 44a has plural photoelectric elements such as photo diodes arranged in plural arrays. The imaging unit 44 converts light received by the imaging surface 44a photoelectrically, and stores signal charge according to a light amount of received light at respective pixels. The signal charge is converted into a voltage signal by an amplifier, and is read. The voltage signal is an image signal, which is output by the imaging unit 44 to the analog processing unit 45.

The imaging unit 44 is a full-color type. Micro color filters of three colors of B, G and R are disposed on the imaging surface 44a, and assigned to respectively pixels. In FIG. 7, spectral distribution of the micro color filters is illustrated. White light emitted by the first light source units 31 is separated by the micro color filters into B, G and R light components. An example of arrangement of the micro color filters is Bayer arrangement.

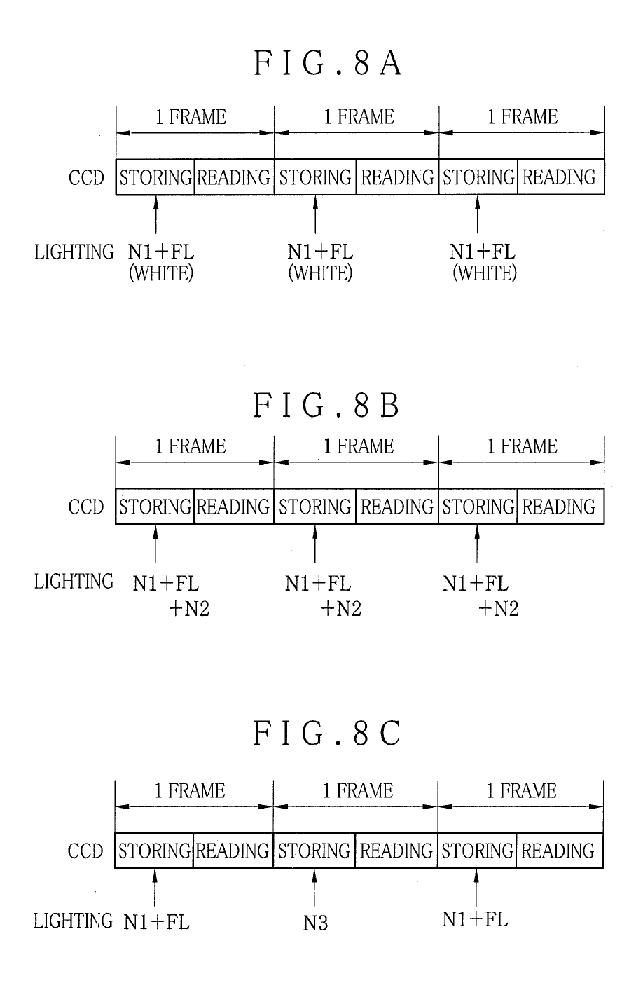

In FIGS. 8A, 8B and 8C, the imaging unit 44 in the normal imaging mode carries out storing and reading, and stores signal charge within a period of acquisition of one frame in the storing, and reads the stored signal charge in the reading. In FIG. 8A, the laser diode LD1 is turned on in the normal imaging mode according to the sequence of the storing. White light obtained from the narrow band light N1 and the fluorescence FL is applied to an object of interest. Reflected light from the object is received by the imaging unit 44. In the imaging unit 44, white color is separated in the color separation by a micro color filter. Reflected light corresponding to the narrow band light N1 is received by the B pixels. A G component in the fluorescence FL is received by the G pixels. An R component in the fluorescence FL is received by the R pixels. The imaging unit 44 sequentially outputs image signals B, G and R of one frame according to a frame rate with pixel values of pixels of B, G and R according to the sequence of the reading. The operation of the imaging is repeated in the course of the normal imaging mode.

In the vessel enhancement mode, the second light source unit 32 in addition to the first light source units 31 is turned on according to the sequence of the storing, as illustrated in FIG. 8B. When the first light source units 31 are turned on, the white light (N1+FL) in combination of the narrow band light N1 and fluorescence FL is applied to an object of interest, in a manner similar to the normal imaging mode. When the second light source unit 32 is turned on, the narrow band light N2 and the white light (N1+FL) are applied to the object of interest.

The light after addition of the white light and the narrow band light N2 is separated by the B, G and R micro color filters in the imaging unit 44 in a manner similar to the normal imaging mode. The B pixels in the imaging unit 44 receive the narrow band light N2 in addition to the narrow band light N1. The G pixels receive a G component in the fluorescence FL. The R pixels receive an R component in the fluorescence FL. In the vessel enhancement mode, the imaging unit 44 sequentially outputs the image signals B, G and R according to the frame rate in the sequence of the reading. Those steps of the imaging are repeated in the course of the vessel enhancement mode.

In the oxygen saturation monitoring mode, the first light source units 31 are turned on according to the sequence of the storing as illustrated in FIG. 8C. In response, the white light (N1+FL) is applied to an object of interest in a manner similar to the normal imaging mode. In a second frame, the first light source units 31 are turned off. The third light source unit 33 is turned on to apply narrow band light N3 to the object of interest. Also in the oxygen saturation monitoring mode, the imaging unit 44 outputs image signals B, G and R according to the frame rate in the sequence of the reading.

In the oxygen saturation monitoring mode, the white light (N1+FL) and the narrow band light N3 are used alternately for emission in a manner different from the normal imaging mode and the vessel enhancement mode. Image signals B, G and R corresponding to the white light are output at a first frame. Image signals B, G and R corresponding to the narrow band light N3 are output at a second frame. Information according to the image signals B, G and R changes for each of the frames in correspondence with the illumination light. This sequence of imaging is repeated in the oxygen saturation monitoring mode.

In FIG. 3, the analog processing unit 45 includes a correlated double sampling circuit (CDS), an automatic gain control device (AGC) and an A/D converter (all not shown). The correlated double sampling circuit processes an image signal of an analog form from the imaging unit 44 in a correlated double sampling, and eliminates electric noise due to resetting a signal charge. The automatic gain control device amplifies the image signal after eliminating the noise in the correlated double sampling circuit. The A/D converter converts the amplified image signal from the automatic gain control device into a digital image signal of gradation steps according to a predetermined number of bits. The digital image signal is input to the processing apparatus 12.

A controller 56 is incorporated in the processing apparatus 12. The imaging control unit 46 is connected with the controller 56, is synchronized with a base clock signal from the controller 56, and outputs a drive signal to the imaging unit 44. The imaging unit 44 outputs an image signal to the analog processing unit 45 at a predetermined frame rate according to the drive signal from the imaging control unit 46.

The processing apparatus 12 includes a digital signal processor 57 (DSP), an image processing unit 58, a frame memory 59 and a display control unit 60 in addition to the controller 56. The controller 56 has a ROM, RAM and the like. The ROM stores a control program and various data required for the control. The RAM is a working memory for loading the control program. The CPU runs the control program to control various elements of the processing apparatus 12.

The digital signal processor 57 receives the image signal from the imaging unit 44. The digital signal processor 57 separates the image signal into image signals B, G and R, and processes those in the pixel interpolation. Also, the digital signal processor 57 processes the image signals B, G and R in signal processing, for example, white balance correction.

The frame memory 59 stores the image data output by the digital signal processor 57 and the processed data processed by the image processing unit 58. The display control unit 60 reads the processed image data from the frame memory 59, converts this into a video signal such as a composite signal and component signal, and outputs the video signal to the display panel 14.

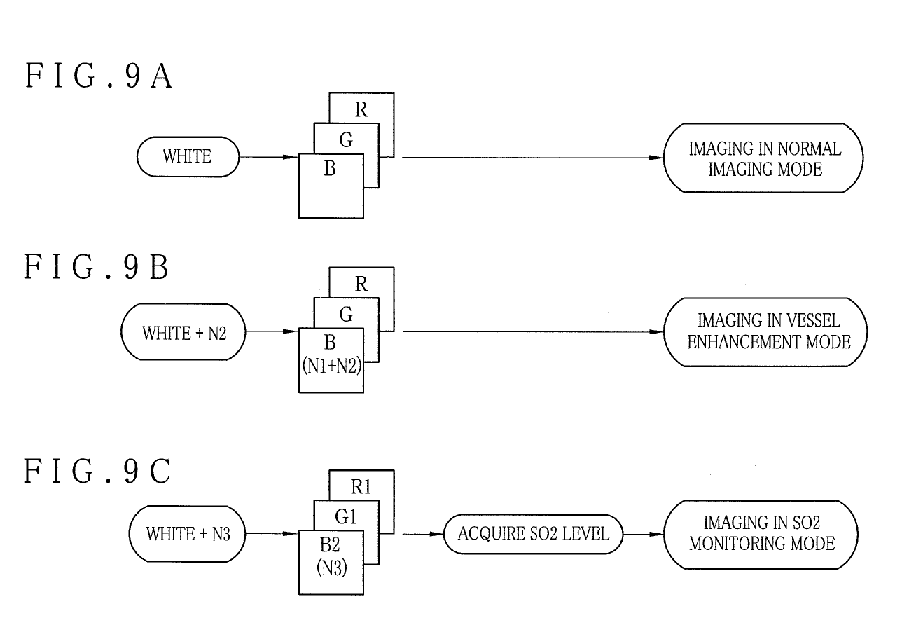

In FIG. 9A, the image processing unit 58 in the normal imaging mode creates an image for the normal imaging according to the image signals B, G and R after color separation in the digital signal processor 57. The image is displayed on the display panel 14. The image processing unit 58 updates the image at each time that the image signals B, G and R in the frame memory 59 are updated.

In FIG. 9B, the image processing unit 58 in the vessel enhancement mode creates an image for the vessel enhancement according to the image signals B, G and R. The image signal B in the vessel enhancement mode has information of a B component in the white light (narrow band light N1 and part of fluorescence FL) and information of the narrow band light N2. Thus, surface vessels are imaged at high contrast. It is known that there is a characteristic pattern of vessels in a tumor or other lesions, for example, density of surface vessels is higher in lesions than in normal tissue. Accordingly, it is preferable to image the surface vessels clearly in the vessel enhancement mode for the purpose of diagnosing benign and malignant conditions of a tumor and the like.

To enhance the surface vessels, areas of the surface vessels are detected according to the image signal B, and are processed for image processing such as edge enhancement. The processed image signal B is combined with a full-color image created from the image signals B, G and R. As a result, the surface vessels are reliably enhanced. It is possible to process areas of images of moderately deep vessels and deep vessels similarly in the vessel enhancement. To this end, the areas of the moderately deep vessels and deep vessels are extracted from the image signal G in which information of those is remarkably contained. The extracted areas are processed for the edge enhancement. The image signal G after the edge enhancement is combined with the full-color image generated from the image signals B, G and R.

The vessel-enhanced image of an object of interest is a full-color image similar to a regular image because of the B, G and R image signals. However, blue density of the image signal B in the vessel enhancement mode is higher than that of the image signal B in the normal imaging mode. It is possible in the vessel enhancement to correct the vessel-enhanced image with color balance near to that of the regular image for the normal imaging mode. The image processing unit 58 generates the vessel-enhanced image at each time that the B, G and R image signals in the frame memory 59 are updated.

Other methods of creating a display image in the vessel enhancement can be used. For example, the object of interest may be displayed in pseudo color representation. An image is created only from the image signals B and G without use of the image signal R, to assign the image signal B to the B and G channels, and assign a signal associated with the image signal G to the R channel of the display panel 14.

In FIG. 9C, the image processing unit 58 in the oxygen saturation monitoring mode processes the image signals G1 and R1 acquired by use of white light and the image signal B2 acquired by use of the narrow band light N3, for obtaining an oxygen saturation level. The pixel value of the image signal B2 includes information of a blood amount or density in addition to the oxygen saturation level. For higher precision, it is necessary to separate information of the blood amount from the pixel value of the pixel signal B2. The image processing unit 58 arithmetically operates between the image signals B and R with high correlation to the blood amount, and separates information of the blood amount from the oxygen saturation level.

Specifically, the image processing unit 58 refers to pixel values of the image signals B2, G1 and R1 at the same points, and obtains a ratio B/G of the pixel value of the image signal B2 to the pixel value of the image signal G1, and a ratio R/G of the pixel value of the image signal R1 to the pixel value of the image signal G1. The image signal G1 is used as a reference signal of a brightness level of an object of interest for normalizing the pixel values of the image signals B2 and R1. Then the oxygen saturation level after removing information of the blood amount is determined according to an initially prepared table of a correlation between the ratios B/G and R/G, the oxygen saturation level and the blood amount. A full-color image according to the image signals B2, G1 and R1 is processed in the color conversion according to the determined value of the oxygen saturation level, so that a display image for the oxygen saturation monitoring mode is created.

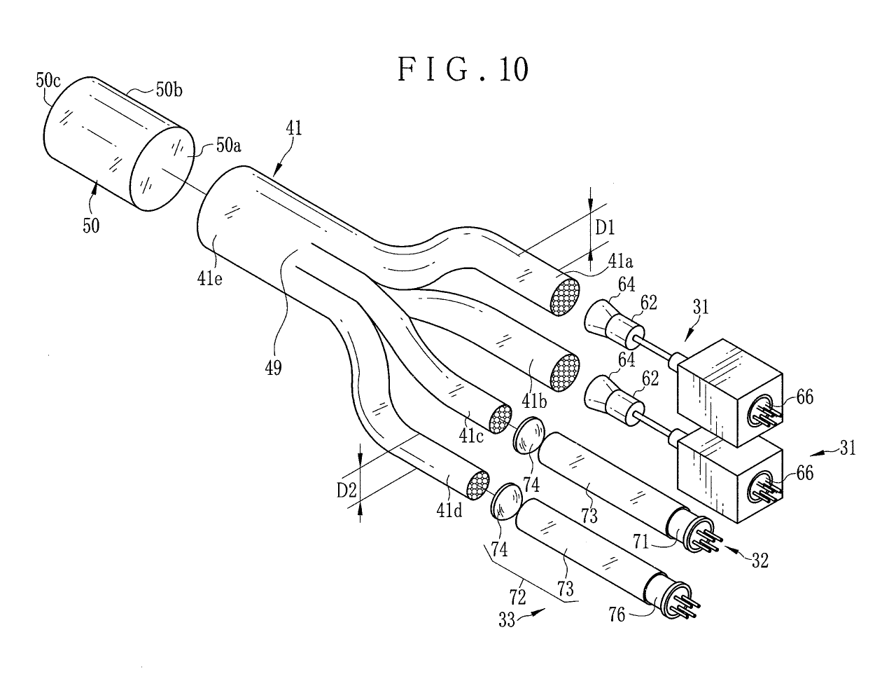

In FIG. 10, the optical routing device 41 in the light source apparatus 13 is a fiber bundle obtained by bundling plural optical fibers in a manner similar to the light guide device 43 of the endoscope 11. All of the optical fibers are collected at the output fiber end 41e of the optical routing device 41, but are split into four groups at the intermediate routing section 49. The input fiber ends 41a-41d are formed by bundling the optical fibers of each of the groups.

A diameter D1 of the input fiber ends 41a and 41b is set different from a diameter D2 of the input fiber ends 41c and 41d by changing the number of optical fibers bundled respectively. The diameter D1 is larger than the diameter D2. This is because the first light source units 31 have the phosphor 36 in contrast with the second and third light source units 32 and 33 without the phosphor 36. A beam diameter of the light flux of the first light source units 31 associated with the input fiber ends 41a and 41b is larger than that of the second and third light source units 32 and 33 associated with the input fiber ends 41c and 41d. Also, one more reason for the difference is that the first light source units 31 emitting white light for the normal imaging should be constructed for a higher light amount than the second and third light source units 32 and 33 for special light imaging.

Specifically, a diameter of the light guide device 43 of the endoscope 11 is approximately 2 mm. A diameter of the output fiber end 41e of the optical routing device 41 is also approximately 2 mm. The diameter D1 of the input fiber ends 41a and 41b is approximately 1.0-1.4 mm. The diameter D2 of the input fiber ends 41c and 41d is approximately 0.5-0.8 mm.

The light homogenizer 50 is disposed at the output fiber end 41e of the optical routing device 41. The light homogenizer 50 homogenizes distribution of light of the plural colors from the light source units 31-33. The light homogenizer 50 is a light guide rod of a cylindrical shape in the optical axis direction, and formed from quartz glass or other transparent material. The light homogenizer 50 includes an entrance end face 50a, an exit end face 50c, and a reflective interface 50b or peripheral surface. The entrance end face 50a receives light exited from the optical routing device 41. The reflective interface 50b tubularly extends in the optical axis direction from the entrance end face 50a, and propagates the incident light in the optical axis direction by the total internal reflection. The exit end face 50c emits the axially directed light.

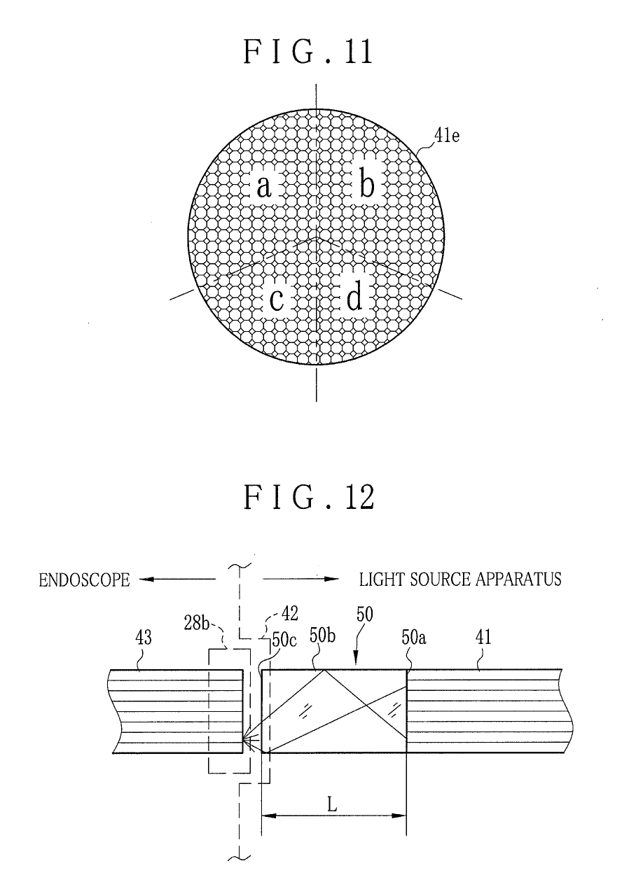

In FIG. 11, optical fibers positioned in respectively the areas a, b, c and d in the output fiber end 41e indicated by the phantom lines are assigned to the optical path of the input fiber ends 41a-41d. The optical fibers for the input fiber ends 41a-41d are distributed at the output fiber end 41e with local unevenness. Light incident through the input fiber ends 41a-41d is propagated in each of the optical fibers. There is no transmission of light between adjacent optical fibers. White light generated by the first light source units 31 exits from the output fiber end 41e through the areas a and b. Narrow band light N2 generated by the second light source unit 32 exits through the area c. Narrow band light N3 generated by the third light source unit 33 exits through the area d. In short, the light of the plural colors is distributed unevenly with the different areas. As a result, distribution of light amounts of light of the colors is uneven in a cross section of the light beam exited from the output fiber end 41e.

In FIG. 12, the light homogenizer 50 propagates incident light by the total reflection with the reflective interface 50b. An entrance position and exit position of the light are changed in a cross section perpendicular to the optical axis direction. This is effective in homogenizing a light amount of the light in a cross section of the light beam directed to the light guide device 43, because the unevenness of light of the plural colors at the output fiber end 41e is canceled. The light homogenizer 50 and the output fiber end 41e are thermally welded to one another in an unified form.

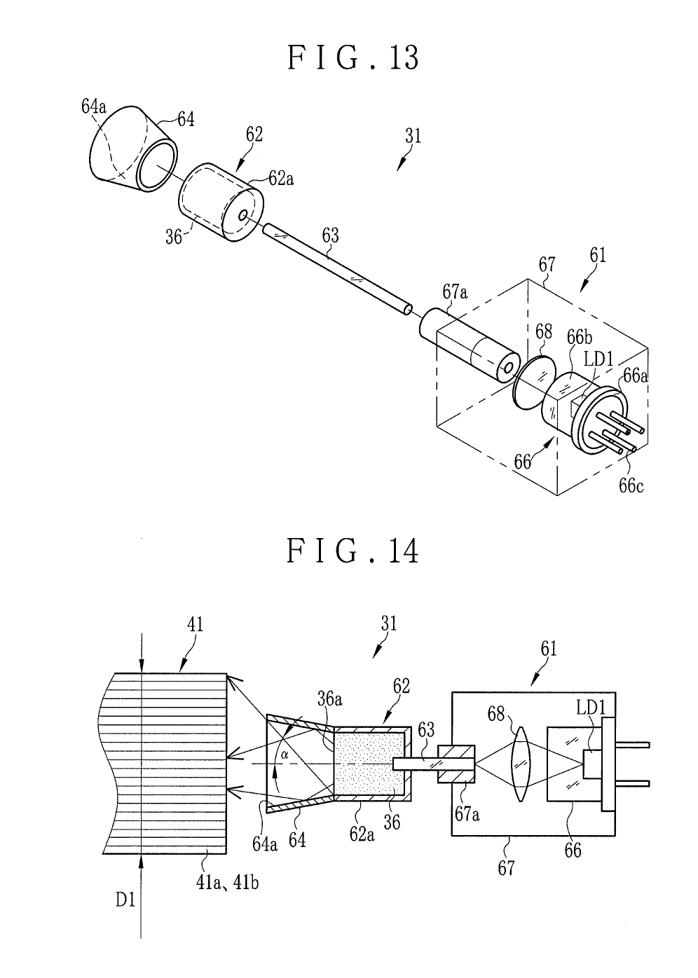

In FIGS. 13 and 14, each of the first light source units 31 includes a laser source 61, a wavelength converter 62 of phosphor conversion, fiber optics 63 of a single fiber, and a divergence angle corrector 64. The fiber optics 63 guide the light from the laser source 61 to the wavelength converter 62. The divergence angle corrector 64 is mounted on an end of the wavelength converter 62. The laser source 61 is in a receptacle form, and includes a light emitting device 66 or laser diode LD1, and a source housing 67 for containing the light emitting device 66. A fiber coupler 67a is provided in the source housing 67 for connection of one end of the fiber optics 63. A condenser lens 68 is incorporated in the source housing 67.

The light emitting device 66 includes a support disk 66a as a stem, the laser diode LD1, a transparent cap 66b, and leads 66c or lines. The laser diode LD1 is a semiconductor chip (light emitting element), and attached to a surface of the support disk 66a. The transparent cap 66b is a cylindrical part of resin, and covers the laser diode LD1. The leads 66c extend from a second surface of the support disk 66a.

The laser diode LD1 includes a P layer of a P type semiconductor and an N layer of an N type semiconductor mounted on the P layer with an active layer, which emits laser light according to laser oscillation. The laser light generally travels straight, but is diverging light of which a diameter of a beam shape increases conically from the emission point. The laser light is condensed by the condenser lens 68 at the entrance end of the fiber optics 63.

An exit end of the fiber optics 63 is coupled with the wavelength converter 62. A container 62a for protection is a cylindrical part of a light-tight property, and filled with the phosphor 36 to constitute the wavelength converter 62. A fiber hole is formed at the center of the phosphor 36 and receives entry of the fiber optics 63. A ferrule (not shown) for connection is mounted on an end of the fiber optics 63, which are entered in the phosphor 36 together.

The phosphor 36 includes phosphor material of a powder form, and binder of resin in which the phosphor material is dispersed and hardened. Emission points of the fluorescence FL upon excitation are disposed on the entirety of the exit end face of the phosphor 36 because of the dispersion. Laser light transmitted through the phosphor 36 is diffused owing to the effect of light scattering of the binder, so that the emission points of the fluorescence FL are disposed on the entirety of the exit end face.

Light emitted by the phosphor 36 is diverging light traveling from the emission points conically in a manner similar to the laser diode LD1. An area of the emission point and a divergence angle of the phosphor 36 are larger than those of the laser diode LD1.

The phosphor 36 has an exit end face 36a. The divergence angle corrector 64 is disposed downstream of the wavelength converter 62 for correcting a divergence angle of the light emitted by the exit end face 36a. The divergence angle corrector 64 is formed cylindrically from opaque material, and reduces the divergence angle by limiting passage of the diverging light from the phosphor 36. An inner surface 64a of the divergence angle corrector 64 is coated with reflective material, and is a mirror surface for the divergence angle corrector 64 to operate as a reflector. The light is reflected by the inner surface 64a and is propagated in the optical axis direction. A loss in the light transmission is low because the absorption of light is low with the inner surface 64a.

Inclination angles of the divergence angle corrector 64 with respect to the transverse direction and optical axis direction are predetermined in consideration of the diameter D1 of the input fiber ends 41a and 41b. The diameter and the inclination angles are so determined that a light spot diameter of a light beam from the first light source units 31 to the input fiber ends 41a and 41b is substantially equal to the diameter D1 of the input fiber ends 41a and 41b.

A divergence angle is determined according to a numerical aperture (NA) of optical fibers as elements of a fiber bundle such as the optical routing device 41, the light guide device 43 of the endoscope 11, and the like. As is well-known in the art, an optical fiber includes a core with a high refractive index, and a cladding with a low refractive index. The incident light upon entry in the optical fiber travels in the optical axis direction. It is necessary to make light incident upon an entrance end face to satisfy the condition of total reflection for the purpose of the propagation.

NA is a value of ability of an optical fiber for condensing light, and is defined as NA=sin θmax wherein θmax is a maximum reception angle. NA increases according to an increase in the maximum reception angle θmax. If an incident angle of light incident in the optical fibers is equal to or smaller than the maximum reception angle θmax, total reflection occurs on an interface between the core and cladding in the optical fiber. The incident light travels in the optical axis direction. If the incident angle becomes larger than the maximum reception angle θmax, the incident light cannot be propagated, because of passage without total reflection. There occurs a loss in the transmission of light. To reduce the loss in the light, the divergence angle corrector 64 regulates the divergence angle of the light beam from the first light source units 31 equal to or lower than the maximum reception angle θmax.

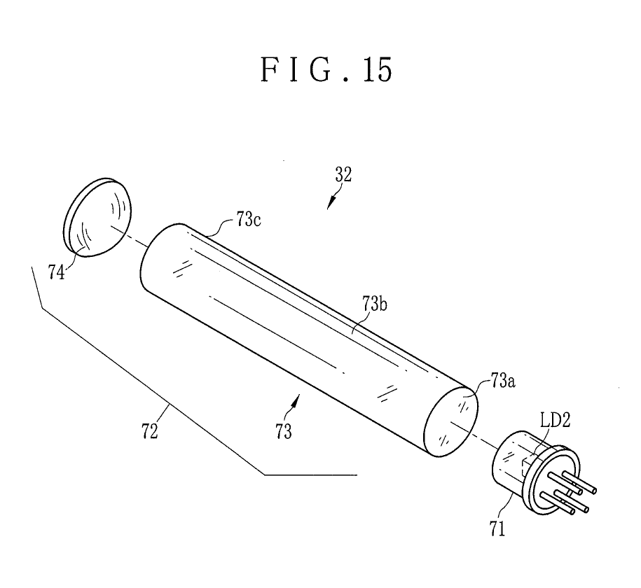

In FIG. 15, the second light source unit 32 includes a light emitting device 71 and a divergence angle corrector 72. The light emitting device 71 has the laser diode LD2, and is structurally the same as the light emitting device 66 in the first light source units 31. The divergence angle corrector 72 includes a light homogenizer 73 and a hemispherical lens 74. The light homogenizer 73 is structurally the same as the light homogenizer 50 of the first light source units 31, but has a size different from the light homogenizer 50, and is a light guide rod of a cylindrical shape in the optical axis direction, and is formed from quartz glass or other transparent material. The light homogenizer 73 can be called a light pipe or light tunnel, and extends longitudinally in the optical axis direction. A shape of the light homogenizer 73 as viewed in a cross section is circular.

The light homogenizer 73 includes an entrance end face 73a, an exit end face 73c, and a reflective interface 73b or peripheral surface. The entrance end face 73a receives the light beam exited from the laser diode LD2. The reflective interface 73b is cylindrical and tubularly extends in the optical axis direction from the entrance end face 73a. The exit end face 73c emits the axially directed light beam. A diameter of the light homogenizer 73 is constant from the entrance end face 73a to the exit end face 73c. The reflective interface 73b extends in parallel with the optical axis direction. The entrance end face 73a of the light homogenizer 73 is thermally welded to a distal end face of the light emitting device 71, to combine the light homogenizer 73 with the light emitting device 71 in an unified form. Owing to the thermal welding, a contact area of a surface of the optical path with air is smaller than a comparative structure in which small spaces are defined between portions of an optical path without unified form. A specific value of the diameter of the light homogenizer 73 is equal to the diameter D2 of the input fiber end 41c, for example, approximately 1.0 mm.

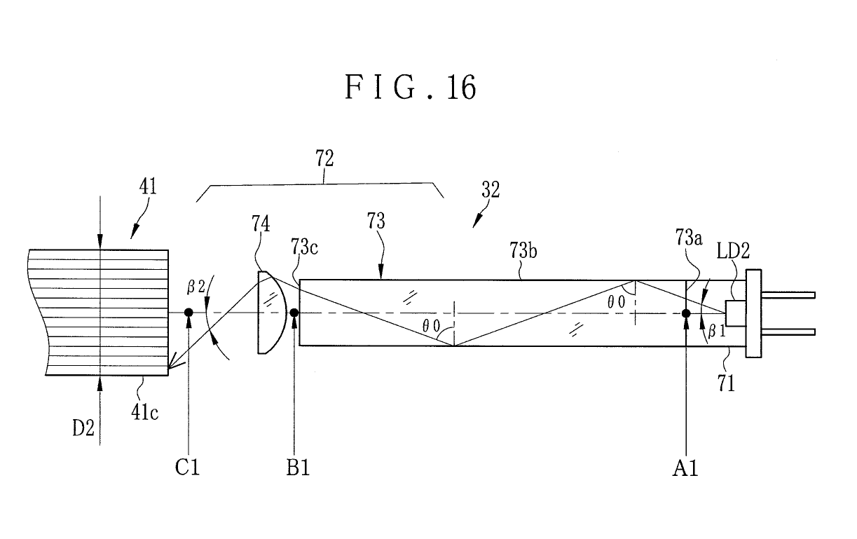

In FIG. 16, the light homogenizer 73 directs a light beam in the optical axis direction by total internal reflection on the reflective interface 73b after entry through the entrance end face 73a. A light component included in the light beam becomes incident upon a point on the optical axis at the entrance end face 73a, and then exits from the exit end face 73c at a point disposed in the periphery offset from the optical axis. Similarly, various light components included in the light beam pass an incident position and an exit position different from the incident position in a radial direction. In short, the light components in the light beam are diffused in the radial direction in the light homogenizer 73. The exit end face 73c of the light homogenizer 73 emits a light beam of which irradiance distribution is flat in the radial direction.

As the light homogenizer 73 has the constant diameter, the divergence angle is maintained in the passage of the light beam. A reflection angle θ0 at which the rays in the light beam are reflected internally by the reflective interface 73b depends upon the incident angle of the rays incident upon the entrance end face 73a. The reflection angle θ0 is constant in a range extending to the reflective interface 73b.

The hemispherical lens 74 is disposed directly downstream of the exit end face 73c of the light homogenizer 73. The hemispherical lens 74 is a convex lens of which a first surface is plane, and a second surface is hemispherical and is opposed to the exit end face 73c. A diameter of the hemispherical lens 74 is approximately 1.5 times as large as that of the light homogenizer 73. For example, the light homogenizer 73 has the diameter of approximately 1 mm, the hemispherical lens 74 having the diameter of approximately 1.5 mm. The hemispherical lens 74 refracts incident light, and enlarges a divergence angle of the light beam from a first divergence angle β1 to a second divergence angle β2.

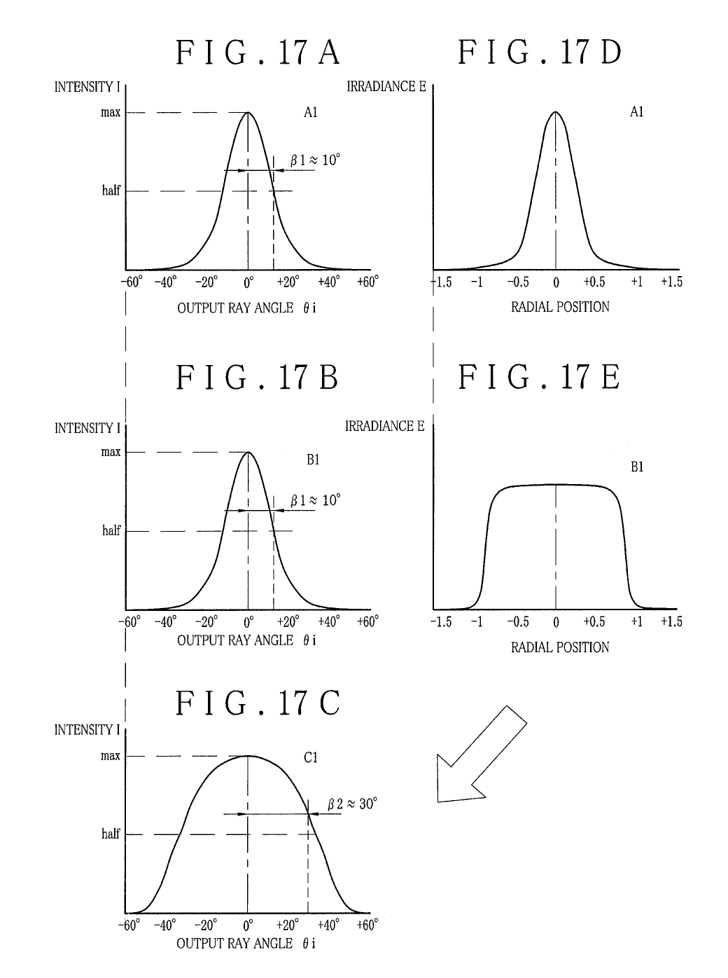

Details of operation of the light homogenizer 73 and the hemispherical lens 74 are described by referring to FIGS. 17A-17E. In FIGS. 17A and 17D, results of simulation are illustrated in relation to intensity distribution and irradiance distribution of a light beam from the laser diode LD2 upstream of the light homogenizer 73, namely at the point A1 in FIG. 16. In FIGS. 17B and 17E, results of simulation are illustrated in relation to intensity distribution and irradiance distribution of the light beam from the laser diode LD2 downstream of the light homogenizer 73 and upstream of the hemispherical lens 74, namely at the point B1 in FIG. 16. In FIG. 17C, a result of simulation is illustrated in relation to intensity distribution of the light beam from the laser diode LD2 downstream of the hemispherical lens 74, namely at the point C1 in FIG. 16. Note that the simulation is carried out by setting diameters of the light homogenizer 73 and the hemispherical lens 74 two times as long as the actual diameters to obtain results illustrated in FIGS. 17A-17E.

In FIGS. 17A, 17B and 17C, intensity distribution of the light beam at the points A1, B1 and C1 is illustrated in graphs. In a manner similar to FIG. 46, the horizontal axis represents an output ray angle θi. The vertical axis represents intensity I. The intensity I is a radiation flux (in lumen) per unit solid angle (in sr or steradian). A unit of the intensity I is lumen/sr. In FIGS. 17D and 17E, irradiance distribution of the light beam at the points A1 and B1 is illustrated as a result of arithmetic operation from the intensity distribution at the points A1 and B1. The horizontal axis represents a position (in millimeter) in a radial direction perpendicularly intersecting with the optical axis. The vertical axis represents an irradiance E. The irradiance E is a radiation flux per unit area, and expressed in lumen/m².

Intensity distribution of the light beam of the laser diode LD2 at the point A1 is the same as that illustrated in FIG. 46. This is a Gaussian distribution in which the intensity I drops abruptly from the peak of the curve with a steep inclination. Also, irradiance distribution at the point A1 is Gaussian in a manner similar to the intensity distribution.

In the irradiance distribution at the point A1, the light beam from the laser diode LD2 has a high center light amount at the peak, but has a peripheral light amount relatively lower than the center light amount. The light homogenizer 73 is effective in increasing the peripheral light amount of the laser diode LD2 by the function of the diffusion. In the irradiance distribution at the point B1, the exit end face 73c of the light homogenizer 73 emits the light beam of the irradiance distribution of a top-hat shape in which the irradiance is constant in the radial direction.

As the diameter of the light homogenizer 73 is constant, the divergence angle of the light beam is maintained in the course of passage. The intensity distribution at the point B1 is equal to that at the point A1 disposed upstream of the light homogenizer 73. The light homogenizer 73 enlarges an area of emission of the light beam in the radial direction to homogenize the irradiance without changing the intensity distribution. Thus, a peripheral light amount increases relatively to a center light amount in comparison with the point before the entry.

The light beam of the top-hat irradiance distribution enters the hemispherical lens 74 after the increase in the peripheral light amount through the light homogenizer 73. A light component incident upon the hemispherical lens 74 on the optical axis is passed straight. However, other light components incident upon a peripheral portion of the hemispherical lens 74 in the radial direction are refracted. A radius of curvature of the hemispherical lens 74 is constant. A refractive index of portions of the hemispherical lens 74 increases according to a distance of the portions from the optical axis in the radial direction. According to the highness in the refraction of a light component through the hemispherical lens 74, its output ray angle θi after exit from the hemispherical lens 74 increases as a high angle component. As a result, high angle components increase after exiting according to highness in the peripheral light amount of light through the periphery of the hemispherical lens 74. In the second light source unit 32, the light homogenizer 73 increases the peripheral light amount. As illustrated in the intensity distribution at the point C1, the intensity distribution near to a top-hat distribution with an increased high angle component is obtained in comparison with the intensity distribution of a Gaussian shape at the point B1 before incidence upon the hemispherical lens 74.

As the high angle component increases in the intensity distribution at the point C1, the divergence angle 132 becomes as large as approximately 30 degrees as a half width at half maximum (approximately 60 degrees as a full width at half maximum).

In the second light source unit 32, a correction amount of the divergence angle corrector 72 is so determined that the divergence angle β2 is substantially equal to the divergence angle α of exit of the first light source units 31. Specifically, a radius of curvature and a diameter of the hemispherical lens 74 are determined to obtain the correction amount as a target value.

The divergence angles are maintained also in the passage through the optical routing device 41, the light homogenizer 50, and the light guide device 43 of the endoscope 11. As illustrated in FIG. 18, the divergence angle β of the light from the second light source unit 32 (β2 in FIG. 16) is set equal to the divergence angle α of the light from the first light source units 31 in relation to exit of the light from each one of the optical fibers 81 in the light guide device 43. Thus, the light spot diameter SDα of the light from the first light source units 31 can be set equal to the light spot diameter SDβ of the light from the second light source unit 32 at the object of interest SB. If the light spot diameter SDβ is different from the light spot diameter SDα, color unevenness may occur due to an overlapped condition between those. The divergence angle corrector 72 sets the divergence angle β equal to the divergence angle α to prevent such color unevenness, because the light spot diameter SDβ is set equal to the light spot diameter SDα.

For the third light source unit 33, the second light source unit 32 is repeated but with a difference in that a light emitting device 76 or laser diode LD3 of FIG. 10 is provided in place of the light emitting device 71. Elements related to the divergence angle corrector 72 are designated with identical reference numerals.

The operation of the embodiment is described now. For diagnosis, the endoscope 11 is connected to the processing apparatus 12 and the light source apparatus 13. A power source for the processing apparatus 12 and the light source apparatus 13 is turned on to start up the endoscope system 10.

The elongated tube 16 of the endoscope 11 is entered in a gastrointestinal tract of a body, to start imaging of an object of interest. In the normal imaging mode, the first light source units 31 are turned on in FIG. 8A. Narrow band light N1 from the laser diode LD1 and the fluorescence FL from the phosphor 36 are mixed together, so that white light is applied to the object of interest.

In FIG. 10, white light emitted by the first light source units 31 enters the input fiber ends 41a and 41b of the optical routing device 41. In FIG. 11, white light traveled from the input fiber ends 41a and 41b is unevenly distributed at the output fiber end 41e. However, the light homogenizer 50 homogenizes the distribution of the light amount of the white light as illustrated in FIG. 12. The white light without unevenness in the light amount in the cross section of the light beam enters the light guide device 43 of the endoscope 11. The white light is transmitted by the light guide device 43 and applied to an object of interest in the gastrointestinal tract by the lighting windows 22.

In FIGS. 8A and 9A, the imaging unit 44 images the object of interest illuminated by the white light (N1+FL). The digital signal processor 57 generates image signals B, G and R. In the normal imaging mode, the image processing unit 58 creates a display image according to the image signal B, G and R. The display control unit 60 converts the image for normal imaging into a video signal, and drives the display panel 14 to display the image. Those steps are repeated in the normal imaging mode.

For the imaging of the vessel enhancement, the input interface 15 is operated to change over to the vessel enhancement mode, so that the processing apparatus 12 starts a function of the vessel enhancement.

In the vessel enhancement mode, the second light source unit 32 is turned on in addition to the first light source units 31 in FIG. 8B, to apply the white light (N1+FL) and the narrow band light N2 to the object of interest. A light beam of the narrow band light N2 from the laser diode LD2 is converted by the light homogenizer 73 in a flat irradiance distribution of a top-hat shape at the point B1. See FIGS. 16 and 17E. Then the light beam passes the hemispherical lens 74 to enlarge a divergence angle with the intensity distribution at the point C1. See FIGS. 16 and 17C. Thus, the divergence angle of the narrow band light N2 from the second light source unit 32 is set equal to that of white light from the first light source units 31. After this, the light beam of the narrow band light N2 becomes incident upon the input fiber end 41c of the optical routing device 41.

The white light and the narrow band light N2 enter the input fiber ends 41a, 41b and 41c of the optical routing device 41, travel to the output fiber end 41e, and become incident upon the light homogenizer 50. Light amount distribution of the white light and the narrow band light N2 is homogenized, before entry in the light guide device 43 of the endoscope 11. The white light and the narrow band light N2 pass through the light guide device 43 and are applied to an object of interest in the gastrointestinal tract through the lighting windows 22.

In FIGS. 8B and 9B, the imaging unit 44 images an object of interest illuminated by the white light (N1+FL) and the narrow band light N2. The digital signal processor 57 generates image signals of B, G and R. In the vessel enhancement mode, the image processing unit 58 generates a display image for the vessel enhancement according to the B, G and R image signals in a manner similar to the normal imaging mode. The display control unit 60 converts the image for the vessel enhancement mode into a video signal, and drives the display panel 14 to display the image. Those steps are repeated in the vessel enhancement mode. Surface vessels are imaged and displayed at high contrast, because the image signal B is created by use of the white light and the narrow band light N2.

In the vessel enhancement mode, the white light and narrow band light N2 emitted by the first and second light source units 31 and 32 are used. The first and second light source units 31 and 32 are corrected by the divergence angle correctors 64 and 72 to set their divergence angles equal to one another. In FIG. 18, full areas of the light beam spots of the white light and narrow band light N2 to the object of interest SB are overlapped on one another. Color unevenness can be reduced.

Also, the divergence angle is enlarged by the divergence angle corrector 72 in driving only the second light source unit 32 without driving the first light source units 31. A large area of view can be obtained, as a light beam spot of the narrow band light N2 can be larger than comparative lighting without the divergence angle corrector 72. A difference in the light amount between the center and periphery in the light beam spot can be decreased by enlarging the divergence angle. An image of high visual recognition can be obtained.

For the oxygen saturation monitoring, the input interface 15 is operated to change over to the oxygen saturation monitoring mode, so that the processing apparatus 12 starts a function of the oxygen saturation monitoring.

In the oxygen saturation monitoring mode, the first light source units 31 and the third light source unit 33 are turned on alternately with one another from frame to frame as illustrated in FIG. 8C. The white light (N1+FL) and the narrow band light N3 are applied to an object of interest alternately.

The white light and the narrow band light N3 enter the input fiber ends 41a, 41b and 41d of the optical routing device 41, travel to the output fiber end 41e, and become incident upon the light homogenizer 50. Light amount distribution of the white light and the narrow band light N3 is homogenized, before entry in the light guide device 43 of the endoscope 11. The white light and the narrow band light N3 pass through the light guide device 43 and are applied to an object of interest in the gastrointestinal tract through the lighting windows 22.

In FIGS. 8C and 9C, the imaging unit 44 sends first and second image signals to the digital signal processor 57 according to the use of the white light (N1+FL) and the narrow band light N3. The digital signal processor 57 generates image signals B1, G1 and R1 according to the first image signal formed by use of the white light, and generates an image signal B2 according to the second image signal formed by use of the narrow band light N3. The image processing unit 58 carries out arithmetic operation of the image signals B2, G1 and R1 to determine an oxygen saturation level from which information of a blood amount is separated. A full-color image according to the image signals B1, G1 and R1 is converted in the color conversion according to the oxygen saturation level, and forms a display image for the oxygen saturation monitoring.

The first and third light source units 31 and 33 are used in the oxygen saturation monitoring mode. In the third light source unit 33, the divergence angle of the narrow band light N3 is enlarged by the divergence angle corrector 72 in a manner similar to the second light source unit 32. The light spot diameter is equal between the white light and the narrow band light N3 from the first and third light source units 31 and 33. No color unevenness occurs in the display image. In the oxygen saturation monitoring mode, image signals according to the white light and narrow band light N3 are acquired in a frame sequential manner unlike the vessel enhancement mode. Arithmetic operation between the images is carried out from the image signals according to those, so that reliability in the arithmetic operation can be higher by correcting the color unevenness of the white light and narrow band light N3.

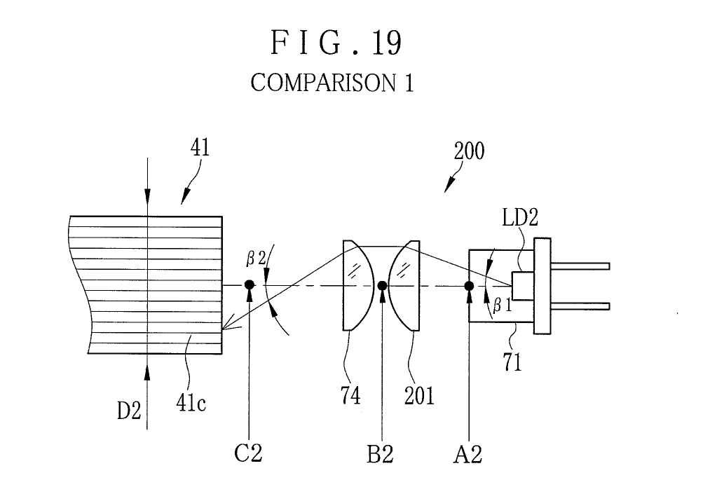

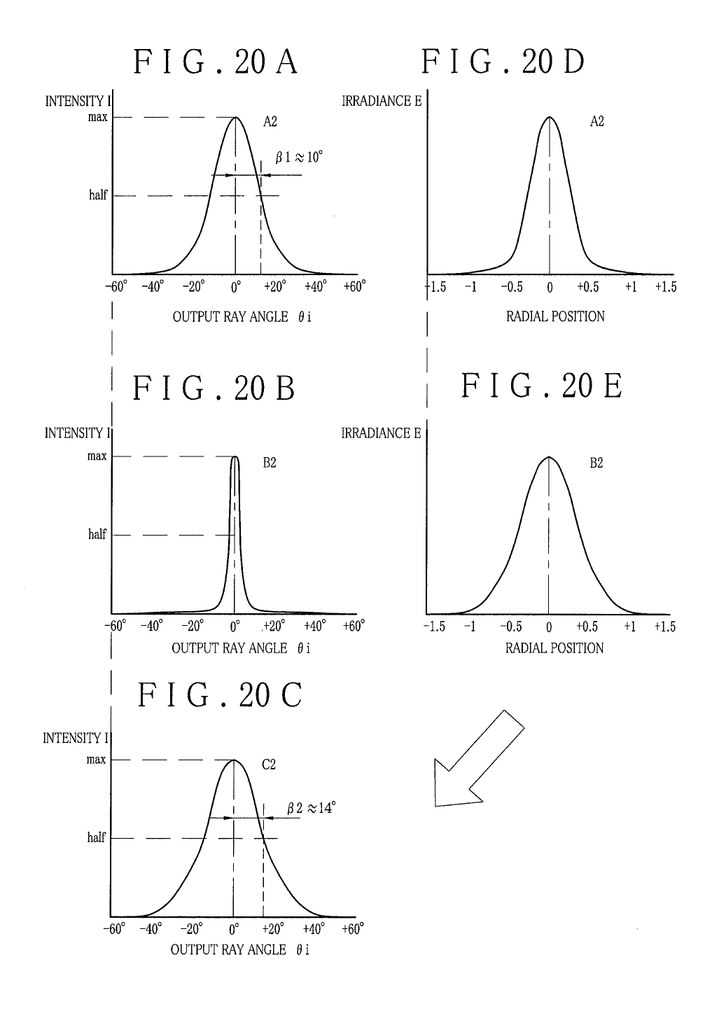

As has been described heretofore, the divergence angle corrector 72 enlarges the divergence angle of the laser diode. The divergence angle corrector 72 includes the hemispherical lens 74 and the light homogenizer 73 disposed in series. Importance of the combination of the light homogenizer 73 with the hemispherical lens 74 refracting light is described by referring to FIGS. 19 and 20A-20E for Comparison 1.

In Comparison 1, a second light source unit 200 of FIG. 19 is provided. The second light source unit 32 is repeated but with a difference in having a plano-convex lens 201 in place of the light homogenizer 73. Elements similar to those of the embodiment are designated with identical reference numerals.

FIGS. 20A-20C are graphs of intensity distribution of the light beam at points A2, B2 and C2 in FIG. 19. FIGS. 20D and 20E are graphs of irradiance distribution of the light beam at points A2 and B2. In FIGS. 20A and 20D, the intensity distribution and irradiance distribution at the point A2 are the same as those at the point A1 in FIGS. 17A and 17D.

The plano-convex lens 201 has a diameter equal to that of the hemispherical lens 74, and collimates the light beam from the laser diode LD2. In FIG. 20B, the intensity distribution at the point B2 is illustrated. A divergence angle of the light beam exited from the plano-convex lens 201 is as small as zero degree because of the collimation of the light beam. There is no function in the plano-convex lens 201 for diffusing light in a radial direction in the manner of the light homogenizer 73. The irradiance distribution does not become flat at the point B2 in the manner of FIG. 20E even after the collimation of the light. In a manner similar to the irradiance distribution at the point A2 before the entry, the distribution remains Gaussian with a large difference between the peripheral light amount and the center light amount indicating a peak of the irradiance.

The light beam exited from the plano-convex lens 201 enters the hemispherical lens 74 in the irradiance distribution of the Gaussian shape. A light amount of the light beam incident upon the hemispherical lens 74 is high at the optical axis, but low in its peripheral portions. As described above, the hemispherical lens 74 causes light components to travel straight on the optical axis, but refracts light components in the peripheral portions. The light components in the periphery become high angle components in intensity distribution after exiting for contribution to enlargement of the divergence angle.

In the second light source unit 200 of Comparison 1, the peripheral light amount of light incident upon the hemispherical lens 74 is lower than in the second light source unit 32, so that there is a smaller increase in the high angle component after the exit. In the intensity distribution in FIG. 20C, a divergence angle β2 of the light beam exited from the hemispherical lens 74 is approximately 14 degrees in the second light source unit 200. Effect of enlarging the divergence angle with the hemispherical lens 74 in the second light source unit 200 is smaller than in the second light source unit 32. The intensity distribution at the point C2 is nearly Gaussian, as is clearly understood in comparison with the intensity distribution at the point C1 in FIG. 17C. A difference in the light amount between the center and periphery in the light beam spot cannot be reduced in the Gaussian intensity distribution. It is impossible to obtain the effect of the invention to reduce the light amount difference by use of the second light source unit 200 of Comparison 1.

In the comparison, the second light source unit 200 has a two-component structure including the hemispherical lens 74 and the plano-convex lens 201. However, the same problem remains with the second light source unit 200 having a single lens component in which the hemispherical lens 74 and the plano-convex lens 201 are unified.

In the laser diode LD2, the divergence angle of the light beam of the irradiance distribution of the Gaussian shape is enlarged in the present invention. Before entry in the hemispherical lens 74, the irradiance distribution is changed into a top-hat distribution by the light homogenizer 73 for increasing the peripheral light amount. Thus, the divergence angle can be enlarged effectively by the hemispherical lens 74. The intensity distribution after changing the divergence angle is nearly top-hat distribution at the point C1 of FIG. 17C. It is possible to reduce a difference in the light amount between the center and periphery of the light beam spot.

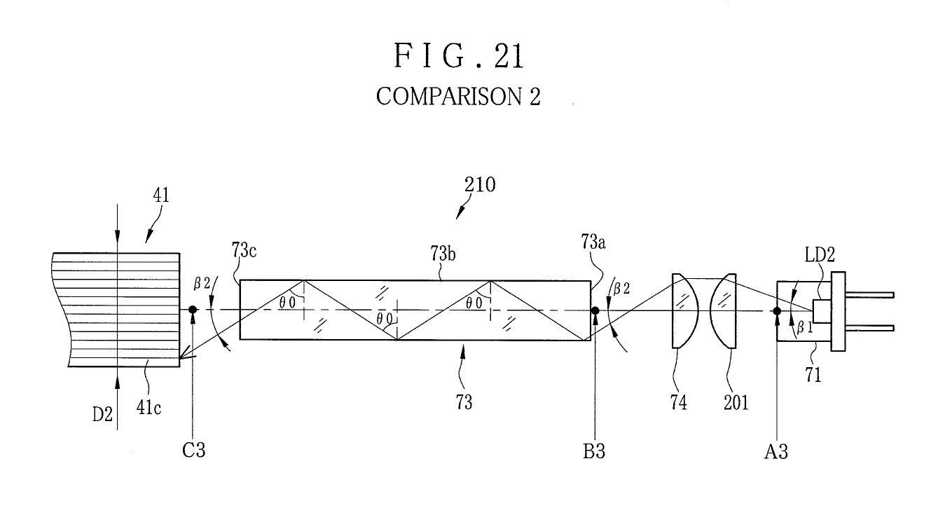

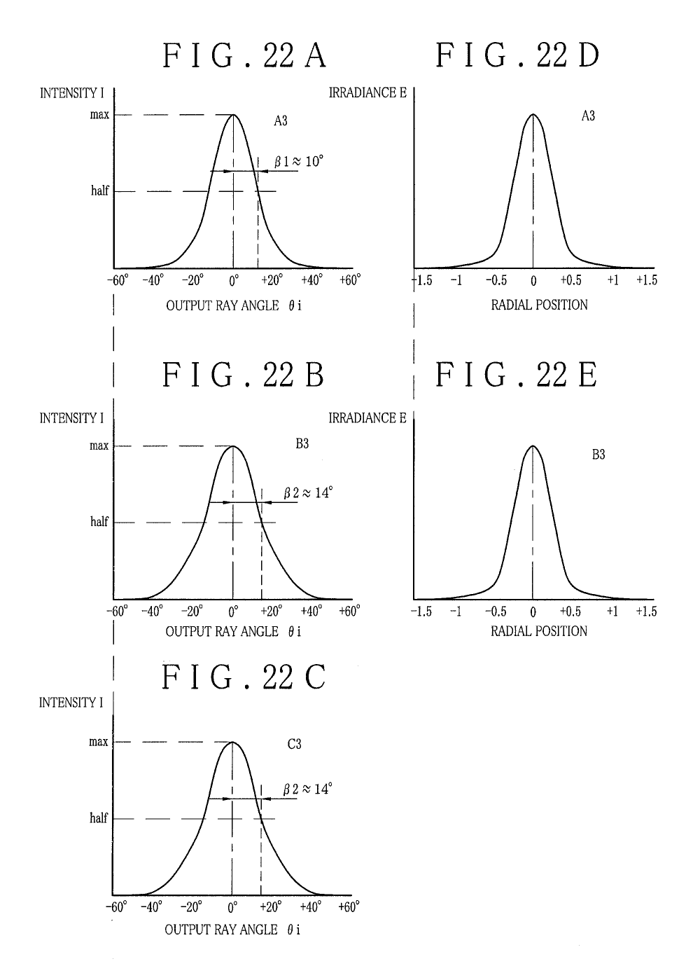

The disposition of the light homogenizer 73 upstream of the hemispherical lens 74 is essentially important in the invention. No effect will be created should the light homogenizer 73 be disposed downstream of the hemispherical lens 74. In FIGS. 21 and 22A-22E, details of an example are illustrated.

In FIG. 21, Comparison 2 is illustrated. A second light source unit 210 has the light homogenizer 73 additionally disposed downstream of the lens optics including the hemispherical lens 74 and the plano-convex lens 201 of Comparison 1. The elements of the second light source unit 32 or 200 are repeated in the second light source unit 210.

FIGS. 22A-22C are graphs of intensity distribution of a light beam at the points A3, B3 and C3 in FIG. 21. FIGS. 22D and 22E are graphs of irradiance distribution of the light beam at the points A3 and B3. In FIGS. 22A and 22D, the intensity distribution and irradiance distribution at the point A3 are the same as those at the point A1 in FIGS. 17A and 17D and those at the point A2 in FIGS. 20A and 20D. In FIG. 22B, the intensity distribution at the point B3 is the same as that at the point C2 in FIG. 20C. In FIG. 22E, the irradiance distribution at the point B3 is the same as that at the point A3 in FIG. 22D. This is because the plano-convex lens 201 and the hemispherical lens 74 do not have a function for diffusing the incident light radially in the manner of the light homogenizer 73.

In the second light source unit 210, a light beam with intensity distribution of the point B3 enters the light homogenizer 73. In the course of passage through the light homogenizer 73, the divergence angle of the light beam is maintained. No change occurs in the intensity distribution between conditions before and after the passage. The intensity distribution at the point C3 in FIG. 22C is the same as that at the point B3 in FIG. 22B. Therefore, no effect of the invention is obtained from the second light source unit 210 of Comparison 2 in a manner similar to the second light source unit 200 of Comparison 1.

In the present embodiment, the light homogenizer 73 is cylindrical with the constant diameter. However, a light homogenizer can be formed with a changing diameter in its longitudinal direction. For example, the light homogenizer 73 may be conically tapered in a form of a frustum of a cone. The entrance end face 73a has a large diameter. The exit end face 73c has a small diameter. The reflective interface 73b is shaped with a gradually decreasing diameter from the entrance end face 73a to the exit end face 73c. A reflection angle θ0 in FIG. 16 decreases at each time of internal reflection of light components on the reflective interface 73b. Accordingly, the divergence angle β of the light beam increases. It is possible to construct the light homogenizer 73 with the function for enlarging the divergence angle by forming the tapered shape in addition to the use of the hemispherical lens 74.

However, a diameter of the light homogenizer 73 must be very small because the divergence angle of a light beam with a small beam diameter is enlarged, such as a laser beam. It is practically difficult to form the light homogenizer 73 in the tapered shape with the small diameter. Consequently, it is preferable to keep the diameter of the light homogenizer 73 constant longitudinally in view of suitability for production. Costs of the parts of the light homogenizer 73 can be low owing to the easy production, as an advantage of the feature of the invention.

A preferred example of the hemispherical lens 74 is a short focus lens having a short focal length, namely having a small radius of curvature. A maximum value of the exit angle of light from the lens increases according to shortness in the focal length (smallness in the radius of curvature). Thus, effect of enlarging the divergence angle can increase according to shortness in the focal length. In FIGS. 17B and 17C for the intensity distribution at the points B1 and C1, the hemispherical lens 74 is the short focus lens with which a divergence angle β1 of approximately 10 degrees of a laser beam is enlarged to a divergence angle β2 of approximately 30 degrees of an exiting light beam by an increase of approximately 20 degrees, as indicated in the half width at half maximum. In consideration of the divergence angle required for lighting in the endoscope, an increase of the angle by which the divergence angle β1 of the laser beam is enlarged can be preferably approximately 10 degrees or more in the short focus lens, as indicated in the half width at half maximum.

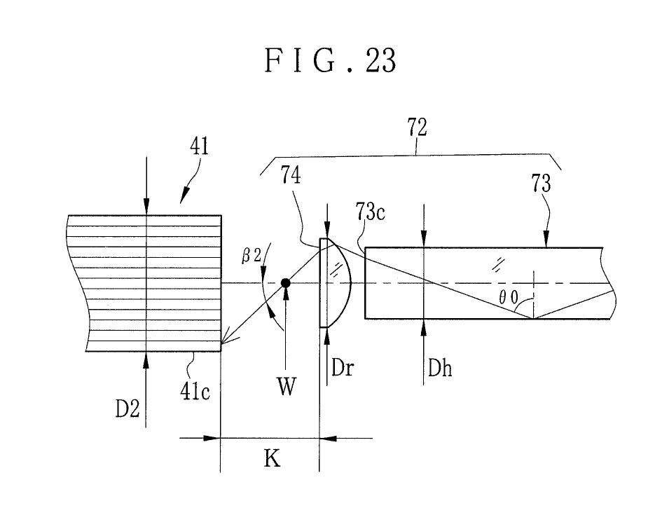

There is an advantage in use of the short focus lens in that an interval K from the exit surface of the hemispherical lens 74 to the input fiber end 41c can be shortened as illustrated in FIG. 23. The light beam exited from the hemispherical lens 74 is converged to a beam waist W with a minimized diameter, and then diverges to enter the input fiber end 41c. As the input fiber end 41c is a fiber bundle, a light spot diameter of the exiting light beam upon entry in the input fiber end 41c is preferably equal to the diameter D2 of the input fiber end 41c for the purpose of incidence of the light upon all of the optical fibers or single fibers in the fiber bundle. The distance from the exit surface of the hemispherical lens 74 to the beam waist W decreases according to a decrease of the focal length. Thus, the interval K can be shortened.

A diameter Dh of the light homogenizer 73 is preferably equal to or smaller than a diameter Dr of the hemispherical lens 74. Should the diameter Dh be larger than the diameter Dr, a loss of light occurs, as light components included in the exiting light beam from the light homogenizer 73 may not be incident upon the hemispherical lens 74. In the present embodiment, the diameter Dr of the hemispherical lens 74 is approximately 1.5 times as much as the diameter Dh of the light homogenizer 73, and is larger than the latter. This is effective in preventing a loss in light.

In consideration of effect of enlarging the divergence angle, the diameter Dr of the hemispherical lens 74 can be preferably set equal to the diameter Dh of the light homogenizer 73. As described above, a portion of the hemispherical lens 74 has a higher refractive index according to a distance from the optical axis in a radial direction. A high angle component increases according to an increase in the peripheral light amount of light incident upon the periphery of the hemispherical lens 74. As the exit end face 73c of the light homogenizer 73 is disposed close to the entrance surface of the hemispherical lens 74. The peripheral light amount of the hemispherical lens 74 increases according to nearness of the diameter Dh of the light homogenizer 73 to the diameter Dr of the hemispherical lens 74. When the diameter Dh of the light homogenizer 73 is equal to the diameter Dr of the hemispherical lens 74, the peripheral light amount is the highest, and the high angle component is the largest. The effect of enlarging the divergence angle becomes the largest. As a result, the diameter Dh is preferably equal to the diameter Dr for this purpose.

In the above embodiment, the hemispherical lens 74 is included in the divergence angle corrector 72. However, lens optics for the hemispherical lens 74 may be a lens without a hemispherical surface, and can be a spherical lens with a larger radius of curvature than that of the hemispherical lens 74. Also, the lens optics can be an aspherical lens without a correctly spherical surface and for the purpose of effect of enlarging the divergence angle as described above.

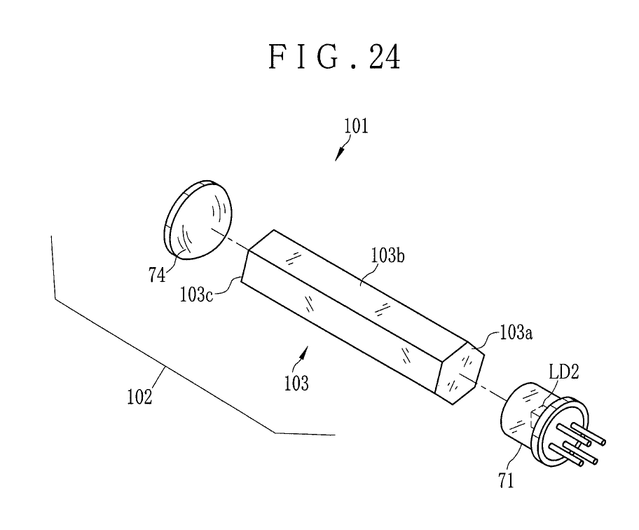

In FIG. 24, another preferred structure of a second light source unit 101 is illustrated. A divergence angle corrector 102 has a function of beam shaping of a beam profile of the light beam from the laser diode LD in addition to the function of enlarging the divergence angle. A light homogenizer 103 is included in the divergence angle corrector 102, and is shaped in a form of a hexagonal prism.

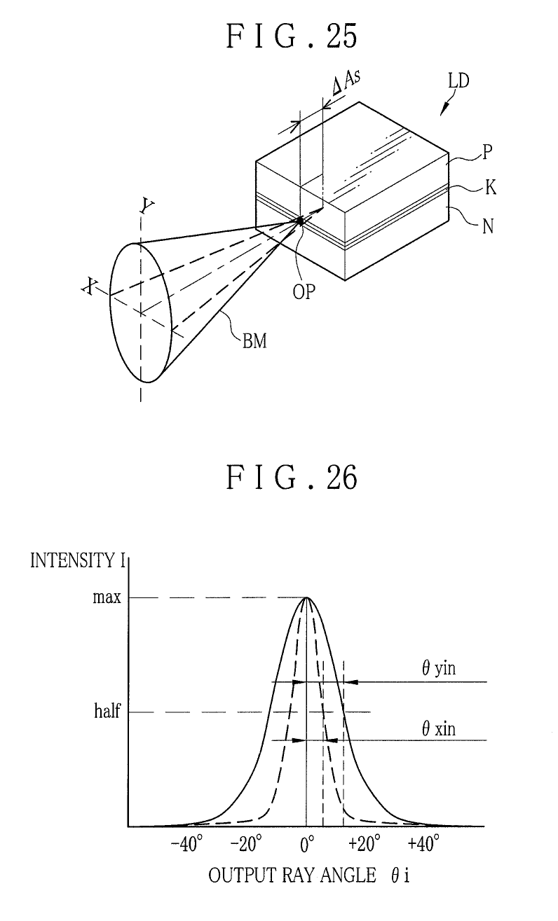

In FIG. 25, the light beam BM emitted by the laser diode LD travels in such a manner that its beam profile is elliptical. The laser diode LD includes a P layer of a P type semiconductor and an N layer of an N type semiconductor mounted on the P layer with an active layer K, which emits laser light beam BM from an emission point OP as diverging light. A first emission point emits light in a horizontal direction or X direction parallel to the active layer K. A second emission point emits light in a vertical direction or Y direction vertical to the active layer K. An astigmatism ΔAs is defined between the first and second emission points in the optical axis direction. Thus, the beam profile of the light beam BM is elliptical with a major axis in the Y direction.

The beam profile of the light beam from the laser diode LD is shaped elliptically. Its intensity distribution is illustrated schematically in FIGS. 17A-17E for the first embodiment. For more precision, the intensity distribution is anisotropic as illustrated in FIG. 26. Intensity is different between an X direction of a phantom line and a Y direction of a solid line. Divergence angles are different between the X and Y directions. A divergence angle θyin of the light beam in the Y direction incident upon the light homogenizer 103 is larger than a divergence angle θxin of the light beam in the X direction. For example, the divergence angle θyin is approximately 12 degrees. The divergence angle θxin is approximately 6 degrees smaller than the divergence angle θyin.

If a beam profile of the light beam is elliptical, a beam spot shape of a light beam spot is also elliptical on the object of interest. The beam spot shape is preferably truly circular, so that beam shaping in a truly circular shape for a beam profile is preferable. To this end, the divergence angle corrector 102 has a function for the beam shaping in the second light source unit 101.

In FIG. 24, the divergence angle corrector 102 includes the hemispherical lens 74 and the light homogenizer 103. The light homogenizer 73 of the above embodiment is repeated but with a difference in a shape of the light homogenizer 103 as viewed in a cross section. Although the light homogenizer 73 is cylindrical, the light homogenizer 103 is in a shape of a hexagonal prism.

In the second light source unit 101, the second light source unit 32 is repeated except for structural differences in the light homogenizer 103. The light homogenizer 103 is formed from quartz glass or other transparent material, and includes an entrance end face 103a, an exit end face 103c, and a reflective interface 103b or peripheral surface. The entrance end face 103a receives the light beam exited from the laser diode LD. The reflective interface 103b extends from the entrance end face 103a, and reflects the light beam in total reflection to direct the same in the optical axis direction. The exit end face 103c emits the axially directed light beam. While the light beam is passed, irradiance of the light beam is homogenized in a radial direction of the light homogenizer 103, for incidence upon the hemispherical lens 74 after conversion into a flat irradiance distribution.

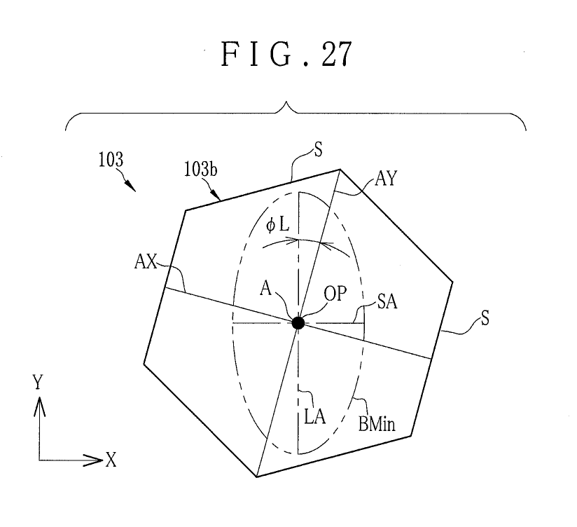

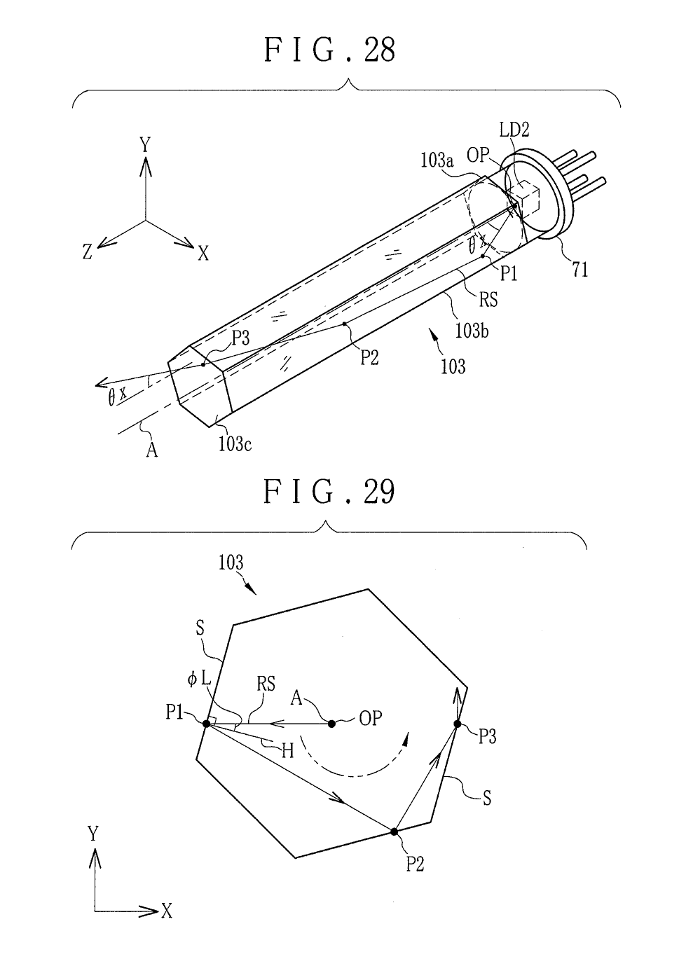

In FIG. 27, the light homogenizer 103 is so disposed relative to the light emitting device 71 that the optical axis A of its center is aligned with the emission center OP of an incident light beam BMin from the laser diode LD2. The incident light beam BMin enters the light homogenizer 103 in an orientation of directing the major axis LA in the Y direction (vertical) and directing the minor axis SA in the X direction (horizontal). Rays in the incident light beam BMin travel radially from the emission center OP.

In FIG. 26, the intensity distribution of the incident light beam BMin of the elliptical beam profile is anisotropic between the X and Y directions. Its divergence angle θyin in the direction of the major axis LA is large. Its divergence angle θxin in the direction of the minor axis SA is small.

The light homogenizer 103 is inclined with an angle φL with respect to the major and minor axes LA and SA about the optical axis A. Let AX be an axis normal (perpendicular) to the optical axis A to pass mid points of two opposite side lines S in the hexagonal section of the light homogenizer 103. Let AY be an axis normal (perpendicular) to the axis AX and to the optical axis A, to pass two opposite vertices of the hexagonal section of the light homogenizer 103. The angle φL is defined between the axis AY and the major axis LA, or between the axis AX and the minor axis SA. In the present example, the angle φL is 15 degrees.

Both of the major and minor axes LA and SA of the incident light beam BMin become non-normal to the side lines S of the hexagon of the reflective interface 103b of the light homogenizer 103 which is inclined. Light components in the incident light beam BMin parallel to respectively the major and minor axes LA and SA become incident upon the side lines S non-normally. Loci of the light components in such directions are described as below.

Let RS be a light component of the minor axis in the incident light beam BMin in FIGS. 28 and 29. The component RS enters the entrance end face 103a of the light homogenizer 103 from the emission center OP. As the optical axis A passes through the emission center OP, the component RS of the minor axis is directed in the X direction parallel to the minor axis SA from the optical axis A in a plane perpendicular to the optical axis A (Z direction). The component RS becomes incident upon one side line S of the hexagon constituting the reflective interface 103b. The reflection of the component RS at a reflection point P1 is total internal reflection. The component RS reflects at the reflection point P1 with the angle φL according to the inclination of the hexagon at the angle φL. This is because the component RS becomes incident non-normally (at an incident angle different from a right angle) with respect to the side line S, namely at the angle φL relative to a normal line H of the side line S. In short, there occurs a twist of the component RS of the minor axis about the optical axis A owing to the reflection at the reflection point P1.

The component RS of the minor axis from the reflection point P1 becomes incident upon a second side line S at a second reflection point P2. As a twist of the component RS has occurred about the optical axis A owing to the reflection at the reflection point P1, the component RS becomes incident upon the reflection point P2 non-normally to the second side line S. The component RS is reflected at a reflection angle equal to or more than 0 degrees with respect to a normal line of the second side line S, and travels toward a reflection point P3. Also, the component RS becomes incident upon the reflection point P3 non-normally to a third side line S. A twist of the component RS occurs at the reflection point P3 about the optical axis A.

The component RS of the minor axis carries out the twist repeatedly about the optical axis A at each of the reflection points P1-P3. As indicated by the curved arrow of the phantom lines in FIG. 29, the component RS of the minor axis travels in the direction of the optical axis A within the light homogenizer 103 by turning around the optical axis A. The travel direction of the component RS of the minor axis changes in the passage through the light homogenizer 103, and thus is different from a travel direction of the entry. If a twist angle of the light as a result of the reflection at the reflection points P1-P3 is 90 degrees about the optical axis A, the travel direction of the component RS of the minor axis parallel to the X direction upon the entry becomes normal (perpendicular) to the Y direction upon the exit.

In FIG. 28, the divergence angle θx of the component RS of the minor axis at the incidence is maintained also in the passage with respect to a plane parallel to the optical axis A, so that the component RS exits with the maintained divergence angle θx. This is because the light homogenizer 103 has a constant diameter from the entrance end face 103a to the exit end face 103c. The reflective interface 103b extends in parallel with the optical axis.

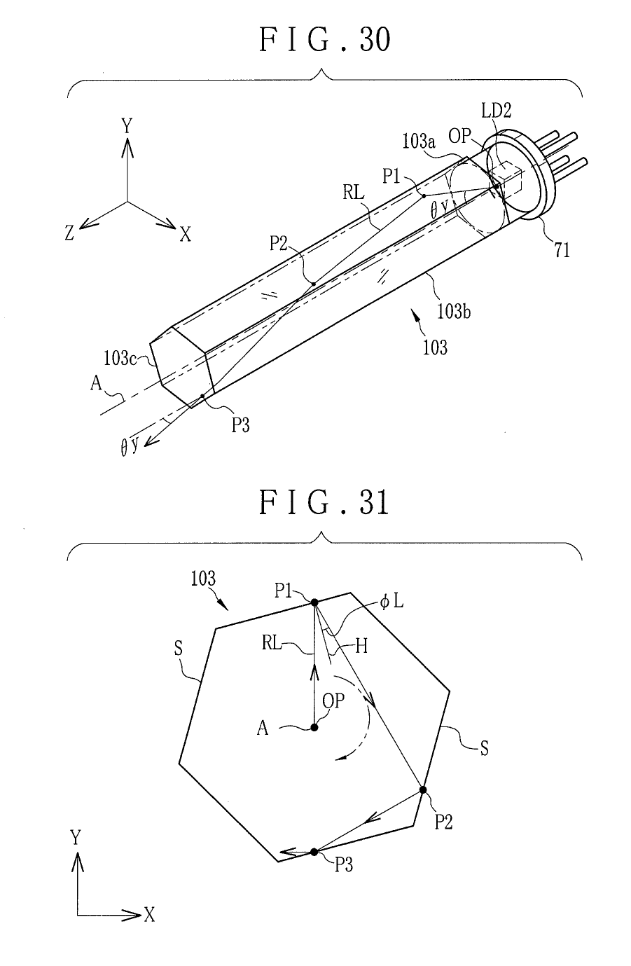

In FIGS. 30 and 31, a locus of the component RL of the major axis normal (perpendicular) to the component RS is illustrated. The component RL is directed in the Y direction from the optical axis A in a plane being perpendicular to the optical axis A. The component RL becomes incident upon the reflection point P1 of the first reflection according to the inclination of the angle φL. This is because the component RL becomes incident non-normally with respect to the side line S, namely at the angle φL relative to the normal line H of the side line S. In short, there occurs a twist of the component RL of the major axis about the optical axis A owing to the reflection at the reflection point P1 in a manner of the component RS.

The component RL of the major axis is repeatedly twisted about the optical axis A by reflection at the reflection points P2 and P3. The component RL travels by turning around the optical axis A as indicated by the curved arrow of the phantom line in FIG. 31. A travel direction of the component RL changes in the passage through the light homogenizer 103 in a manner similar to the component RS. A travel direction of exit of the component RL is different from that upon the incidence. Let a twist angle be 90 degrees about the optical axis A by three reflections of the reflection points P1-P3 in the passage. The component RL of which the travel direction of the incidence is parallel to the Y direction becomes a component parallel to the X direction at the time of exit.

Also, in FIG. 30, the divergence angle Oy is maintained in the passage of light in a plane parallel to the optical axis direction. The component RL of the major axis is exited in a condition of maintaining the divergence angle Oy in a similar manner to the component RS of the minor axis.

The components RL and RS of the major and minor axes in the incident light beam BMin have been described heretofore. Furthermore, intermediate light components in the incident light beam BMin between the components RL and RS are twisted about the optical axis A similarly.

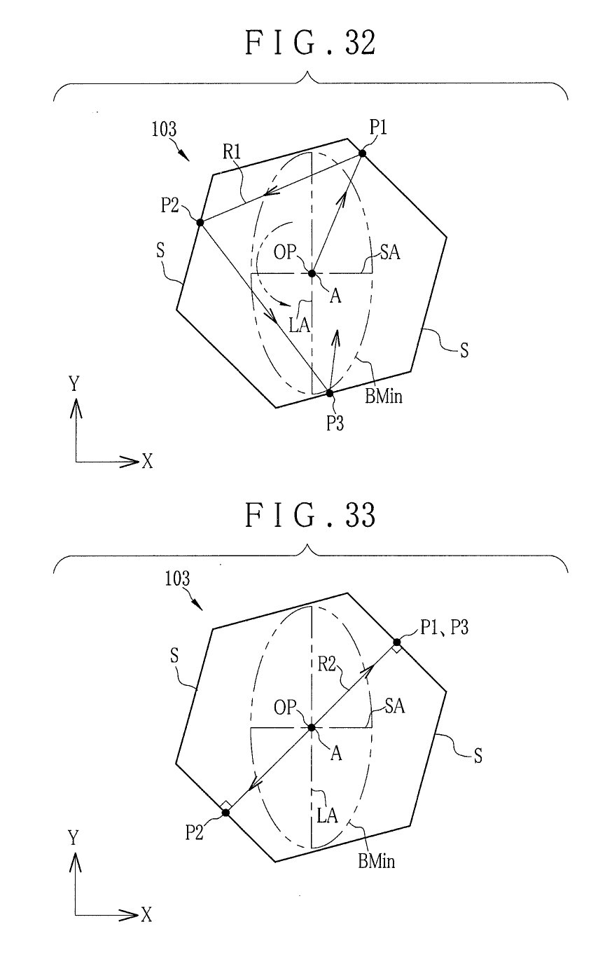

In FIG. 32, an intermediate light component R1 travels in a travel direction between the components RL and RS of the major and minor axes in the incident light beam BMin. The light component R1 becomes incident upon the side line S non-normally in a manner similar to the components RL and RS. A twist occurs at each of the reflection points P1-P3 about the optical axis A to change the travel direction. The light component R1 initially travels for incidence in a direction different from that of the components RL and RS. An incident angle of the light component R1 upon the reflection point P1 of the first reflection is different from that of the components RL and RS. Thus, the light component R1 is twisted at a twist angle and a twist direction different from those of the components RL and RS. The twist direction is indicated by the arcuately curved arrow.

In FIG. 33, there is an intermediate light component R2, which becomes incident upon the side line S normally (perpendicularly), or in parallel with the normal line of the side line S. An incident angle of the light component R2 with respect to the normal line is 0. Its reflection angle at the reflection point P1 is also 0. An origin point of the light component R2 is the optical axis A or emission center OP. As the reflection angle is 0, a locus of the light component R2 after reflection at the reflection point P1 is equal to a locus of its incidence to the reflection point P1. Thus, the light component R2 will not twist about the optical axis A, because reciprocally reflected between the first side line S of the incidence and a second side line S opposite to the first side line S.

Although there occurs no twist of the light R2 about the optical axis A in the light homogenizer 103, components of the incident light beam BMin including the components RL and RS of the major and minor axes travel with twist about the optical axis A, because those components become incident with respect to the side line S non-normally. Also, the angles of the twist are different from one another. In other words, the components included in the incident light beam BMin come to travel in numerous directions in a plane perpendicular to the optical axis A by the internal reflection in the light homogenizer 103.

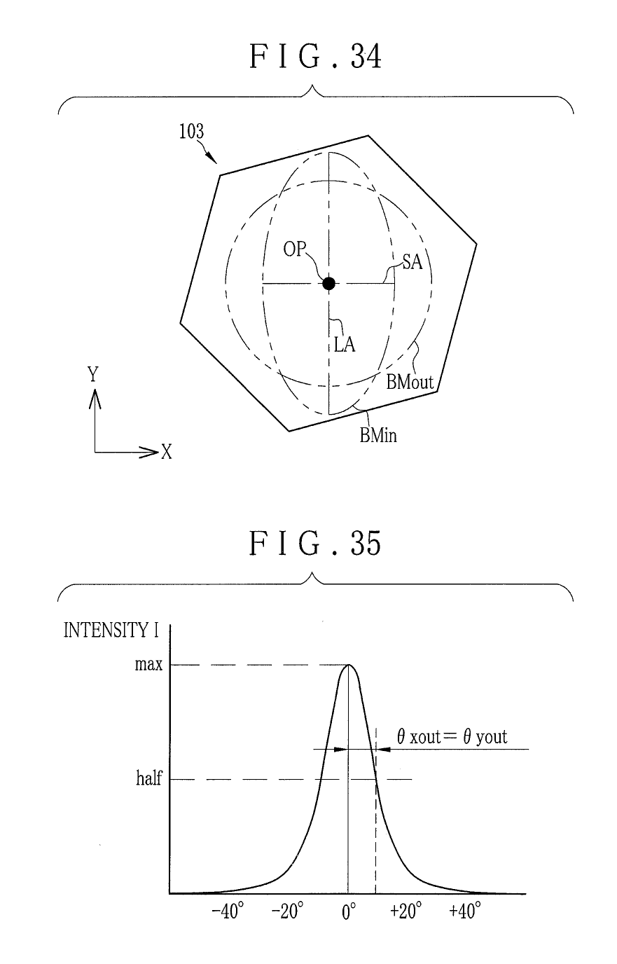

According to the diffusion, the incident light beam BMin is shaped into an exiting light beam BMout by beam shaping in a truly circular beam profile, the incident light beam BMin being shaped elliptically, the exiting light beam BMout exiting through the exit end face 103c. See FIG. 34.

In FIG. 35, intensity distribution of the exiting light beam BMout measured in a condition of measurement for FIG. 26 is illustrated. In FIG. 26, the divergence angle θxin of the incident light beam BMin in the X direction is small in contrast with the divergence angle θyin in the Y direction. However, the light homogenizer 103 operates to enlarge the divergence angle θxout of the exiting light beam BMout in the X direction and reduce the divergence angle θyout of the Y direction. In FIG. 35, the divergence angle θxout becomes equal to the divergence angle θyout. In short, the form of the exiting light beam BMout is shaped in a truly circular manner. Specifically, the divergence angle θyin of the incident light beam BMin in the Y direction (LA) is approximately 12 degrees. The divergence angle Oxin of the incident light beam BMin in the X direction (SA) is approximately 6 degrees. The light homogenizer 103 shapes the incident light beam BMin into the exiting light beam BMout of the circular shape with the divergence angles θyout=θxout=approximately 10 degrees.

The divergence angle of the exiting light beam BMout is enlarged by incidence upon the hemispherical lens 74. As has been described with the first embodiment, the light homogenizer 103 is effective in homogenizing the irradiance in the radial direction. The divergence angle of the exiting light beam BMout can be reliably enlarged by the hemispherical lens 74.

Conventionally, there are known methods of beam shaping, include one in which two cylindrical lenses are used to reduce a size of a light beam in the major axis direction. However, there are drawbacks in this method in that the number of interfaces between air and the lenses is as high as four due to the two lenses. A loss in optical transmission is seriously large with a large Fresnel loss. In contrast with this, in the light homogenizer 103 in the present invention, the number of interfaces between air and optics is two including the entrance and exit faces. A loss in optical transmission can be reduced by use of the single optics of the light homogenizer 103. Furthermore, the function of the beam shaping is disposed together on the light homogenizer for correcting the divergence angle. Thus, the structure can be simplified without an increase in the number of the parts in comparison with originally separate components for a light homogenizer and a portion for the beam shaping.

In the above embodiment, the light homogenizer 103 is associated with the second light source unit having the laser diode LD2. However, the light homogenizer 103 may be associated with the third light source unit having the laser diode LD3.

In FIG. 14, rays in the light beam from the laser diode LD1 in the first light source units 31 are diffused in the phosphor 36. Thus, the rays are emitted by the entire area of the exit end face of the phosphor 36 omnidirectionally. As the divergence angle corrector 64 is shaped circularly in a cross section, a beam profile of the light beam exited from the phosphor 36 is shaped by the divergence angle corrector 64 circularly. Also, a beam profile of the fluorescence excited by the light beam is shaped circularly by the divergence angle corrector 64. The first light source units 31 emit the mixed light including the laser light and the fluorescence as a light beam of a circular beam profile.

In the light source unit including the laser diode and the phosphor such as the first light source unit, the phosphor operates for the beam shaping. The light homogenizer 103 with the function for the beam shaping is effectively used in combination with a light source unit without phosphor, for example, the second and third light source units.

In the light source apparatus having the light source units with and without phosphor, the light homogenizer 103 shapes the light beams from the second and third light source units in a truly circular beam profile. The beam spot shape of the light beams generated by the second and third light source units can be set equal to the beam spot shape of the light beam generated by the first light source unit. Thus, it is possible to reduce color unevenness due to differences in the beam spot shape of the various light source units.

In the above embodiment, the angle φL of the axes AX and AY in the light homogenizer 103 is 15 degrees as illustrated in FIG. 27. However, the angle φL may not be 15 degrees, and can be a value predetermined in a range of 0-60 degrees.

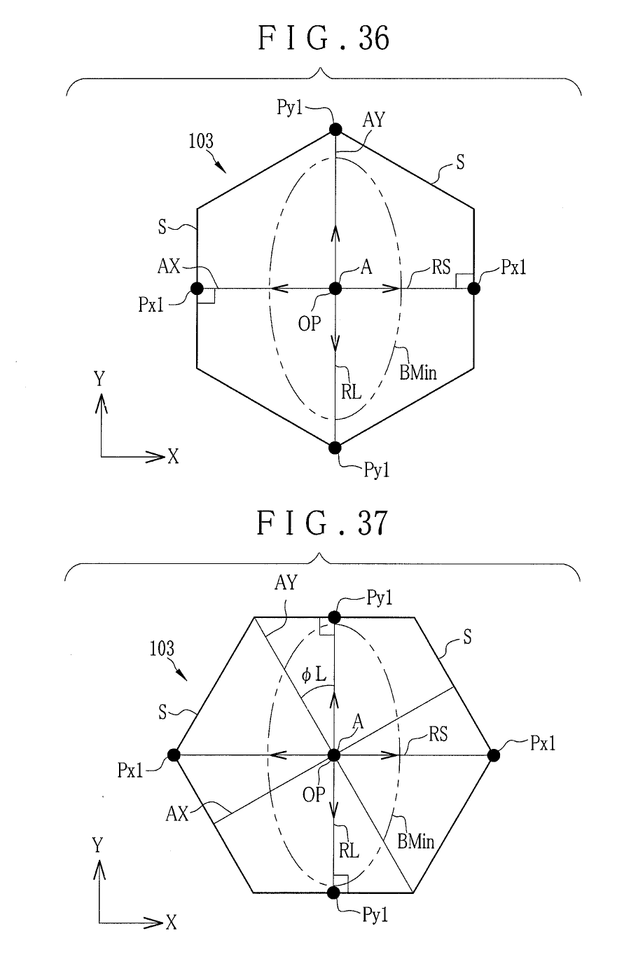

In FIG. 36, an example of φL=0 is illustrated. The component RS of the minor axis in the incident light beam BMin is aligned with the axis AX. The component RL of the major axis in the incident light beam BMin is aligned with the axis AY. The component RS from the optical axis A becomes incident normally (perpendicularly) upon the side line S of the hexagon. A reflection angle at the reflection points Px1 of the first reflection (relative to the normal line of the side line S) is 0. The component RS travels back and forth between the two opposite reflection points Px1. No twist about the optical axis A occurs.

As the component RL of the major axis becomes incident upon vertices of the hexagon, the vertices of the hexagon are first reflection points Py1. As a reflection angle is equal to or more than 0 degrees at the reflection points Py1, a twist about the optical axis A occurs with the component RL. Intermediate components between the components RL and RS of the major and minor axes become incident non-normally (with an angle different from the right angle) relative to the side lines S, and consequently with a twist about the optical axis A. Therefore, a beam profile of the light beam BM is shaped substantially in a truly circular manner, as light components in the incident light beam BMin are diffused on a plane perpendicular to the optical axis A.

In FIG. 37, an example of φL=30 degrees is illustrated. The component RL of the major axis becomes incident normally (perpendicularly) to the side lines S in contrast with the example of FIG. 36. No twist about the optical axis A occurs. However, the component RS of the minor axis becomes incident non-normally (with an angle different from the right angle) to the side lines S, because the vertex of the hexagon is a first reflection point Px1. There occurs a twist about the optical axis A. Intermediate components between the components RL and RS of the major and minor axes become incident with a twist about the optical axis A. Therefore, a beam profile of the light beam BM is shaped substantially in a truly circular manner.

According to results of experiments and simulations, it is found that effect of the beam shaping is obtained if twist occurs about the optical axis A with only at least one of the components RL and RS of the major and minor axes. It is noted that non-normal incidence of those upon the side lines S is remarkably preferable for good effect of the beam shaping. The most preferable example of the angle φL is 15 degrees as illustrated in FIG. 27 as a result of an experiment or simulation.

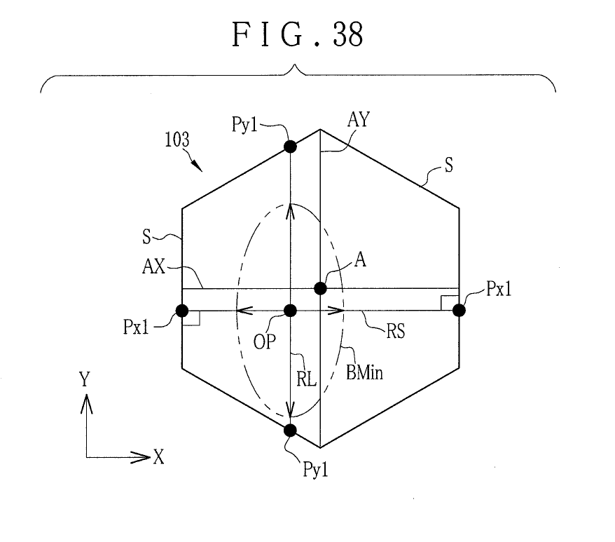

In FIG. 38, the emission center OP of the incident light beam BMin is offset from the optical axis A as a center of the hexagon of the light homogenizer 103. In short, the light homogenizer 103 is non-concentric with the light emitting device 71. Thus, one of the components RL and RS of the major and minor axes can be incident upon the side line S non-normally. Effect of the beam shaping can be obtained as a twist of one of the components RL and RS occurs about the optical axis A. However, there is a shortcoming in that an area of a cross section of the light homogenizer 103 must be enlarged relative to that of the incident light beam BMin in comparison with a structure without offsetting of the emission center OP from the optical axis A. It is preferable to align the emission center OP with the optical axis A, because the shortcoming of the example of FIG. 38 may be serious for guiding the incident light beam BMin of the single light emitting device 71.

In the above embodiment, the form of the light homogenizer in the cross section is hexagonal. However, a light homogenizer of the invention can be formed in a quadrilateral or triangular shape as viewed in a cross section.

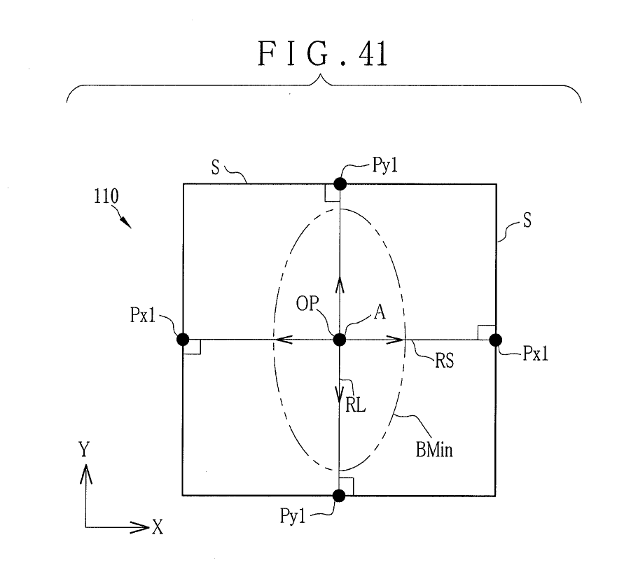

In FIG. 39, a light homogenizer 110 is a light guide rod in a shape of a quadrilateral prism. The light homogenizer 103 is repeated but with a difference in the shape. The light homogenizer 110 is disposed by aligning the optical axis A with the emission center OP of the laser diode LD2. Also, the light homogenizer 110 is disposed so that the two diagonals normal (perpendicular) to one another in the quadrilateral are aligned with respectively the X and Y directions (directions of minor and major axes of the incident light beam BMin). This orientation is defined by rotating a horizontal orientation (see FIG. 41) about the optical axis A by 45 degrees. In the horizontal orientation, the two opposite side lines of the quadrilateral are set in parallel with respectively the X and Y directions.

Accordingly, reflection points Px1 and Py1 of the first reflection of the components RS and RL of the minor and major axes of the incident light beam BMin are vertices of the quadrilateral opposite to one another. The components RS and RL defined radially from the optical axis A enter the reflection points Px1 and Py1, and are twisted about the optical axis A. Thus, the beam shaping of the incident light beam BMin originally in an elliptical shape is carried out to form a truly circular form.

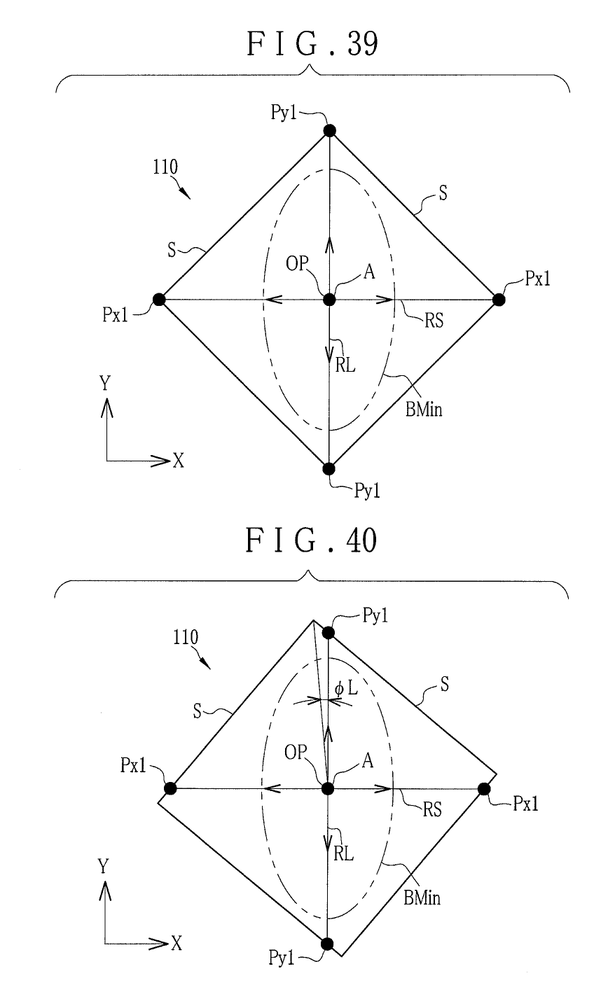

In FIG. 40 for a variant structure, the light homogenizer 110 is inclined with an inclination angle φL about the optical axis A with reference to the original position of FIG. 39. An example of the angle φL is 5 degrees. The orientation of the variant structure is inclined with approximately 40 degrees from the horizontal orientation of FIG. 41. The components RS and RL become incident upon the side lines S non-normally at the reflection points Px1 and Py1 of the first reflection. Effect of the beam shaping can be obtained to shape the incident light beam BMin of an elliptical form into a truly circular form.

When the shape in the cross section is quadrilateral, no good effect of beam shaping can be obtained on the condition of the horizontal orientation in which the two adjacent side lines of the quadrilateral are parallel to respectively the X and Y directions. This is a finding as a result of experiments and simulations. The reason for this is in that no twist about the optical axis A occurs at the reflection point, as both of the components RL and RS of the major and minor axes become normally (perpendicularly) incident upon the side lines S.

When the light homogenizer 110 is oriented horizontally, the components RS and RL of the minor and major axes become incident normally (perpendicularly) to the side lines S at the reflection points Px1 and Py1 of the first reflection. No twist occurs with the components RL and RS of the major and minor axes about the optical axis A. This is the same with the second reflection and so on. The component RS of the minor axis exits in the X direction. The component RL of the major axis exits in the Y direction.

Intermediate light components between the components RL and RS of the major and minor axes in the incident light beam BMin become incident non-normally upon the side lines S, to cause a twist about the optical axis A. However, no twist occurs with the components RL and RS determining the elliptical shape. Thus, the incident light beam BMin will not be shaped in a truly circular form.

Although effect of the beam shaping cannot be obtained by disposition of the light homogenizer 110 in the horizontal orientation, only a small inclination of the light homogenizer 110 from the horizontal orientation can result in good effect of the beam shaping, because the components RL and RS of the major and minor axes can become incident upon the side lines S non-normally. According to results of experiments and simulations, it is found that the most preferable inclination is 45 degrees from the horizontal orientation as illustrated in FIG. 39. It is noted that the light homogenizer 110 may be disposed in the horizontal orientation if the light homogenizer 110 is used for correcting the divergence angle without considering the beam shaping in a manner of the first embodiment.

Also, the emission center OP of the incident light beam BMin can be offset from the optical axis A as a center of the light homogenizer 110 in a manner described with the light homogenizer 103 of the hexagonal form. This is because one of the components RL and RS of the major and minor axes can be incident upon the side lines S non-normally on the condition of an orientation different from the horizontal orientation. Note that there is a shortcoming in that an area of the light homogenizer 103 in the cross section should be larger than a beam profile of the incident light beam BMin in contrast with a condition without the offsetting. The optical axis A is preferably aligned with the emission center OP for the purpose of guiding a light beam from the single light emitting device 71.

In the present embodiment, the quadrilateral is a regular quadrilateral (square). However, a quadrilateral may be a rectangular quadrilateral, parallelogram and the like. Among the various shapes, the regular quadrilateral is the most preferable for the productivity, because the light homogenizer 110 of this shape can be produced the most easily.

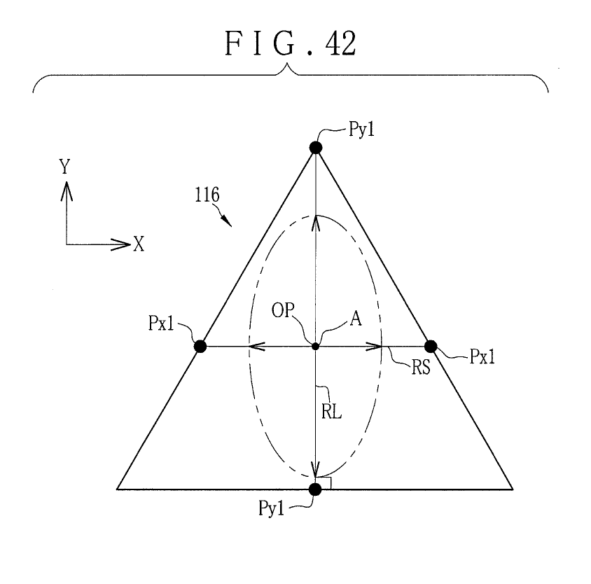

In FIG. 42, a light homogenizer 116 is a light guide rod of a form of a triangular prism. The light homogenizer 103 or 110 is repeated but with a difference in the form. The light homogenizer 116 is positioned by aligning the emission center OP with the optical axis A. A first vertex of the light homogenizer 116 is directed to the upside. A first side line of the light homogenizer 116 opposed to the first vertex is directed to the downside and in parallel with the X direction. This is the horizontal orientation of the light homogenizer 116.

This being so, first and second rays of the component RL of the major axis of the incident light beam BMin becomes incident respectively upon a vertex of the triangle and the reflection point Py1 of the first reflection on the side line S. The second ray of the component RL is normal (perpendicular) to the side line 5, but the first ray travels non-normally in incidence upon at the vertex. Its twist occurs about the optical axis A. The component RS becomes incident upon the two adjacent side lines S. The two adjacent side lines are not normal to the X direction or travel direction of the component RS of the minor axis, which becomes incident upon the reflection points Px1 non-normally. Thus, a twist occurs about the optical axis A. Finally, both of the components RL and RS are twisted about the optical axis A. Effect of beam shaping is obtained to form the beam profile of the incident light beam BMin in a truly circular form.

Although the hexagon and quadrilateral are bilaterally symmetric, the triangle is only rotationally symmetric. It is possible to change the travel direction by twisting about the optical axis A for both of the components RL and RS of the major and minor axes irrespective of an angle of an inclination. Thus, the light homogenizer 116 of the triangular form can be positioned in an orientation different from the horizontal orientation for the purpose of beam shaping. For example, the light homogenizer 116 can be oriented with a rotational shift of 30 or 180 degrees from the horizontal orientation.

Note that the emission center OP can be offset with respect to the optical axis A for the light homogenizer 116 in the triangular shape as viewed in a cross section. The shape of the light homogenizer 116 as viewed in the cross section is a regular triangle according to the embodiment, but can be a rectangular triangle, isosceles triangle and the like. Among the various shapes, the regular triangle is the most preferable for the productivity, because the light homogenizer 116 of this shape can be produced the most easily.

In the above embodiments, the polygon of the form of the cross section of the light homogenizer is the hexagon, quadrilateral and triangle. However, a polygon of the form of the cross section of a light homogenizer can be a pentagon, heptagon or other polygons with eight side lines or more. Note that a light homogenizer in combination with a laser diode has such a small diameter as several mm or less. Thus, a polygon of the form of the cross section of a light homogenizer can be the hexagon or other polygons with six side lines or less.

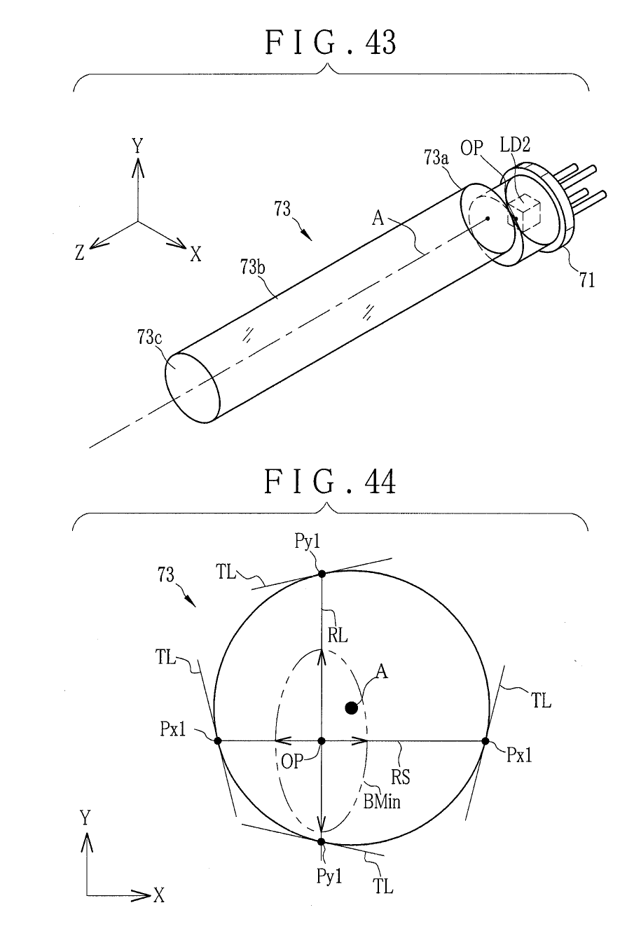

In the above embodiments, the polygonal shape as viewed in a cross section is used for the function of beam shaping. However, the cylindrical shape can be also used for the purpose of beam shaping. In FIGS. 43 and 44 for a variant structure, the light homogenizer 73 of the first embodiment is disposed so that the emission center OP of the laser diode LD2 is offset from the optical axis A of the light homogenizer 73, for the purpose of the beam shaping.

As the form of the light homogenizer 73 in the cross section is truly circular, the reflective interface 73b for the internal reflection is a curved surface.

In FIG. 44, the emission center OP is offset from the optical axis A of the light homogenizer 73 in both of the X and Y directions. In short, the light homogenizer 73 is non-concentric with the light emitting device 71. A reflection point of the component RS of the minor axis in the first reflection is Px1. As a tangential line TL of the reflective interface 73b at the reflection point Px1 is not normal (perpendicular) to the component RS of the minor axis, a reflection angle is defined by reflection of the component RS of the minor axis at the reflection point Px1. A twist of the component RS occurs about the optical axis A to change the travel direction. A reflection point of the component RL of the major axis in the first reflection is Py1. As a tangential line TL of the reflective interface 73b at the reflection point Py1 is not normal (perpendicular) to the component RL of the major axis, a reflection angle is defined by reflection of the component RL of the major axis at the reflection point Py1. Thus, the travel direction changes as twist occurs about the optical axis A. Effect of the beam shaping is obtained to shape the beam for the truly circular beam profile.

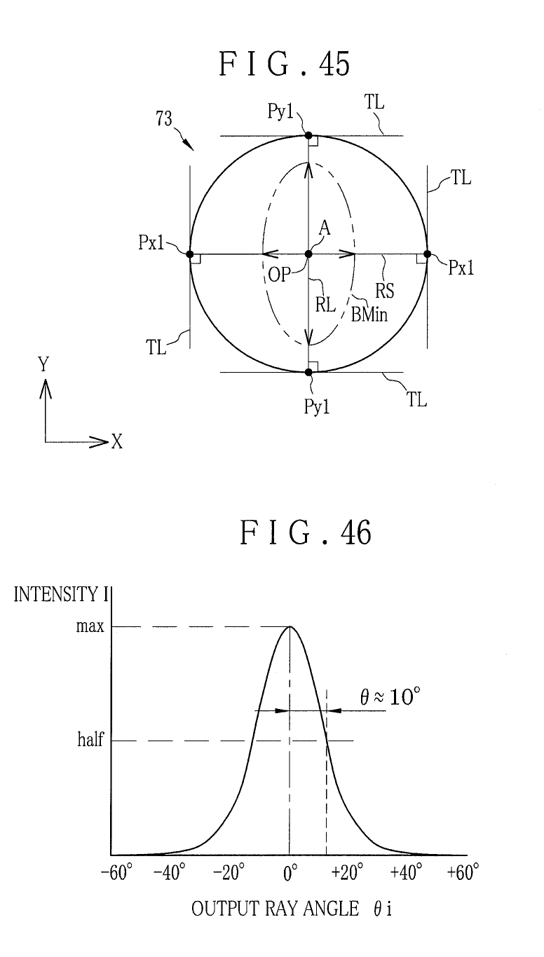

As the light homogenizer 73 has a truly circular shape in the cross section, it is essentially important to offset the emission center OP from the optical axis A for the purpose of effective beam shaping. In FIG. 45, the emission center OP is aligned with the optical axis A of the light homogenizer 73. Tangential lines TL of the reflective interface 73b at the reflection points Px1 and Py1 of the first reflection of the components RS and RL of the minor and major axes are normal (perpendicular) to the components RS and RL. Thus, no twist about the optical axis A occurs. In relation to intermediate light components traveling between the components RL and RS of the major and minor axes, no change occurs in the travel direction. Therefore, there is no effect of beam shaping. A light beam of the elliptical shape exits from the exit end face in the same form as the incident light beam BMin.

In FIG. 44, the emission center OP is offset in both the X and Y directions. However, it is possible to offset the emission center OP only in the X direction of the component RS from the optical axis A of the light homogenizer 73, or only in the Y direction of the component RL. According to results of experiments and simulations, it is found that offsetting in one direction is effective in the beam shaping. It is noted that offsetting in both of the X and Y direction is the most preferable because of high effect in the beam shaping.

In the embodiment, the form of the light homogenizer 73 in the cross section is truly circular. However, a form of a light homogenizer in the cross section in the invention can be elliptical. Also, a form of a light homogenizer in the cross section in the invention can be an eccentric loop constituted by a curve and straight lines.

In the above embodiments, the light homogenizer is the light guide rod. However, a light homogenizer of the invention may be a mirror pipe including a cylindrical tube and a reflective coating applied to an inside of the cylindrical tube for a mirror surface. It is possible in the mirror pipe to reflect the incident light beam to direct this in the optical axis direction. It is possible to obtain effect of homogenizing irradiance of the light beam in the radial direction, and effect of beam shaping by suitably forming the shape of a light homogenizer in the section. Note that the light guide rod is better than the mirror pipe in view of the efficiency in transmitting light, because a loss in the reflection is higher in the mirror reflection than in the total internal reflection.

In the above embodiments, the divergence angle corrector includes the light homogenizer and the lens as two optics. However, a divergence angle corrector may include additional optics together with the light homogenizer and the lens. In the above embodiments, the light beam from the lens in the divergence angle corrector becomes incident upon the entrance end face of the light guide device of the endoscope. However, one or more optics may be added between the lens and the light guide device, so that the light beam can be entered indirectly to the light guide device.

In the above embodiments, the second and third light source units 32 and 33 for narrow band light are used in association with the divergence angle corrector. However, a color or wavelength of a light beam for correction of a divergence angle is not limited, and can be determined suitably. For example, a light source apparatus may include three light sources for generating blue, green and red light fluxes to emit white light. A light homogenizer can be associated with at least one of the three light sources.

In the first light source units 31 of the above embodiments, the light emitting devices of semiconductor are laser diodes with phosphor. However, light emitting devices can be a light emitting diode (LED), electroluminescence (EL) LED, electroluminescence (EL) element, and the like. Furthermore, the first light source units 31 may have a lamp such as a xenon lamp and halogen lamp.

In the above embodiments, the light emitting devices of semiconductor in combination with the divergence angle corrector having the light homogenizer are laser diodes. However, light emitting devices can be a light emitting diode (LED), electroluminescence (EL) LED, electroluminescence (EL) element, and the like. The divergence angle of the beam from the light emitting diode (LED) and EL element is larger than that of the laser diode. It may be necessary to enlarge the divergence angle of the beam from the light emitting diode (LED) and EL element specifically if the light emitting diode (LED) and EL element are used in combination with a light source with a relatively large divergence angle. The feature of the invention is effective for such a structure.

In the above embodiments, images of plural colors are acquired simultaneously. The micro color filters of blue, green and red are used and separate the white light. However, it is possible to use the feature of the invention in a frame sequential imaging, in which a monochromatic imaging unit without color filters is used and acquires color images one after another.

In the above embodiments, the processing apparatus is originally discrete from the light source apparatus. However, the processing apparatus may be combined with the light source apparatus in a single composite apparatus. Also, the endoscope system of the invention may include an ultrasonic endoscope having an ultrasonic transducer in combination with a processing apparatus.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A light source apparatus for supplying a light guide device incorporated in an endoscope with a light beam, comprising:
a light emitting device of semiconductor for generating said light beam;
a light homogenizer for homogenizing irradiance distribution of said light beam in a radial direction; and
a lens, disposed between said light homogenizer and said light guide device, for enlarging a divergence angle of said light beam.

2. Alight source apparatus as defined in claim 1, wherein said light homogenizer is a transparent light guide rod disposed to extend in an optical axis direction of said light beam.

3. Alight source apparatus as defined in claim 1, wherein a diameter of said light homogenizer is constant in an optical axis direction thereof.

4. A light source apparatus as defined in claim 3, wherein said diameter of said light homogenizer is equal to or less than a diameter of said lens.

5. A light source apparatus as defined in claim 3, wherein said diameter of said light homogenizer is equal to a diameter of said lens.

6. A light source apparatus as defined in claim 3, wherein said lens is a short focus lens.

7. A light source apparatus as defined in claim 3, wherein said light emitting device is a laser diode.

8. Alight source apparatus as defined in claim 3, wherein said light beam is narrow band light of a wavelength range of blue.

9. A light source apparatus as defined in claim 3, wherein a beam profile of said light beam is elliptical;
said light homogenizer includes a beam shaping device for beam shaping of said light beam of said elliptical beam profile into a circular beam profile.

10. A light source apparatus as defined in claim 9, wherein said light homogenizer includes:
an entrance end face for receiving incidence of said light beam from said light emitting device;
an exit end face for emitting said light beam toward said lens;
a reflective interface, disposed to extend from said entrance end face to said exit end face, for internally reflecting said light beam and constituting said beam shaping device.

11. A light source apparatus as defined in claim 10, wherein said light beam includes first and second components located in respectively radial directions along major and minor axes of said elliptical form;
said reflective interface twists at least one of said first and second components about said optical axis direction by reflection.

12. A light source apparatus as defined in claim 10, wherein said light beam includes first and second components located in respectively radial directions along major and minor axes of said elliptical form;
said reflective interface includes a first portion for receiving incidence of at least one of said first and second components in a non-normal direction.

13. A light source apparatus as defined in claim 3, wherein said light homogenizer includes:
an entrance end face for receiving incidence of said light beam from said light emitting device;
an exit end face for emitting said light beam toward said lens;
a reflective interface, disposed to extend from said entrance end face to said exit end face, for internally reflecting said light beam.

14. A light source apparatus as defined in claim 13, wherein said light homogenizer is cylindrical and extends in said optical axis direction.

15. A light source apparatus as defined in claim 13, wherein said light homogenizer is in a form of a polygonal prism extending in said optical axis direction, and said reflective interface includes a plane.

16. A light source apparatus as defined in claim 13, wherein said reflective interface has a curved surface at least partially.

17. A light source apparatus as defined in claim 16, wherein said light beam includes first and second components located in respectively radial directions along major and minor axes of said elliptical form;
at least one of said first and second components is reflected by said curved surface at a reflection point, and travels in a direction non-normal to a tangential line of said curved surface at said reflection point.

18. Alight source apparatus as defined in claim 3, wherein a form of said light homogenizer in a cross section transverse to said optical axis direction is circular, eccentrically looped or elliptical, and a center of said form in said cross section is offset from an emission center of said light emitting device.

19. An endoscope system including an endoscope having a light guide device inside, and a light source apparatus for supplying said light guide device with a light beam, comprising:
said light source apparatus including:
a light emitting device of semiconductor for generating said light beam;
a light homogenizer for homogenizing irradiance distribution of said light beam in a radial direction; and
a lens, disposed between said light homogenizer and said light guide device, for enlarging a divergence angle of said light beam.
